SEQUENCE LISTING (2 sequences)

SEQ ID NO: 1 - protein, 311 aa, Artificial Sequence
  Chemically synthesized Rluc
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 2 - protein, 311 aa, Artificial Sequence
  Chemically synthesized Rluc8
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

(19) United States
(12) Patent Application Publication
(10) Pub. No.: US 2013/0272966 A1
(43) Pub. Date: Oct. 17, 2013

(54) RET2IR CONJUGATES, RET2IR CONJUGATE SYSTEMS AND METHODS OF USE THEREOF

(71) Applicant: The Board of Trustees of the Leland Stanford Junior University, (US)

(72) Inventors: Liqin Xiong, Mountain View, CA (US); Jianghong Rao, Sunnyvale, CA (US)

(73) Assignee: The Board of Trustees of the Leland Stanford Junior University, Palo Alto, CA (US)

(21) Appl. No.: 13/802,999

(22) Filed: Mar. 14, 2013

Related U.S. Application Data
(60) Provisional application No. 61/623,224, filed on Apr. 12, 2012.

(51) Int. Cl. C12Q 1/66; A61K 49/00; G01N 33/58; C12N 9/96

(52) U.S. Cl. CPC C12Q 1/66; C12N 9/96; A61K 49/0017; G01N 33/582
USPC 424/9.6; 435/8; 435/188; 435/6.11; 435/7.1

(57) ABSTRACT

Embodiments of the present disclosure include conjugate systems, methods of using conjugate systems, RET₂IR conjugates (also referred to as “BRET-FRET-NIR conjugates”), systems including RET₂IR conjugates, methods of using the RET₂IR conjugates, and the like. In general, embodiments of the present disclosure involve the non-radiative transfer of energy between a bioluminescence donor molecule and a semiconductor polymer and then the non-radiative transfer of energy between the semiconductor polymer and an NIR dye, all without external illumination.

### CLAIM OF PRIORITY TO RELATED APPLICATION

This application claims priority to co-pending U.S. provisional application entitled “RET₂IR CONJUGATES, RET₂IR CONJUGATE SYSTEMS AND METHODS OF USE THEREOF” having Ser. No. 61/623,224, filed on Apr. 12, 2012, which is entirely incorporated herein by reference.

### STATEMENT REGARDING FEDERALLY SPONSORED RESEARCH OR DEVELOPMENT

This invention(s) was made with government support under Grant No.: CA135294 awarded by the National Institutes of Health. The government has certain rights in the invention(s).

### BACKGROUND

Fluorescence imaging has become a powerful technique to visualize biology in its native physiological settings in a living subject, and has been used even in clinics for guiding surgery in cancer patients⁵. One major obstacle encountered with fluorescence imaging is strong autofluorescence arising from living tissues⁶ that significantly compromises imaging sensitivity and specificity. Addressing this technical challenge demands the development of imaging probes that can emit in the near-infrared (NIR) wavelength range where autofluorescence background is significantly decreased, a spectral window where nanoparticle probes offer great potential. One such example is BRET-QD probes based on bioluminescence resonance energy transfer (BRET) between a bioluminescent protein R. reniformis luciferase donor and a quantum dot (QD) acceptor (BRET-QD). BRET-QD has been applied to in vitro detection, protease and nucleic acid sensing, and in vivo lymphatic imaging. However, unfavorable in vivo pharmacokinetics of QDs after systemic injection, concerns regarding their in vivo stability and the intrinsic toxicity of heavy metals of the QD nanoparticles have largely limited their applications in translational research.

### SUMMARY

Embodiments of the present disclosure include conjugate systems, methods of using conjugate systems, RET₂IR conjugates (also referred to as “BRET-FRET-NIR conjugates”), systems including RET₂IR conjugates, methods of using the RET₂IR conjugates, and the like. In general, embodiments of the present disclosure involve the non-radiative transfer of energy between a bioluminescence donor molecule and a semiconductor polymer, and then the non-radiative transfer of energy between the semiconductor polymer and a NIR dye, all without external illumination.

In an embodiment, a conjugate system can include: a BRET-FRET NIR conjugate and a bioluminescence initiating compound, wherein the BRET-FRET NIR conjugate includes a particle that includes a bioluminescence donor molecule and a NIR dye, wherein the particle includes a semiconductor polymer as part of the particle matrix, wherein the bioluminescence donor molecule is attached to the surface of the particle, wherein each of the NIR dyes are disposed within, attached to, or a combination thereof, the surface of the particle, wherein the bioluminescence donor molecule and the bioluminescence initiating compound interact to produce a bioluminescent energy, wherein the bioluminescent energy is absorbed by the semiconductor polymer and the semiconductor polymer produces fluorescent energy in response to the non-radiative transfer of the bioluminescent energy from the bioluminescence donor molecule to the semiconductor polymer, wherein the NIR dye absorbs the fluorescent energy and emits NIR energy in response to the energy transfer of the fluorescent energy from the semiconductor polymer to the NIR dye.

In an embodiment, a conjugate can include: a BRET-FRET NIR conjugate, wherein the BRET-FRET NIR conjugate includes a particle that includes a bioluminescence donor molecule and a NIR dye, wherein the particle includes a semiconductor polymer (e.g., a conducting polymer or semiconducting polymer) as part of the particle matrix, wherein the bioluminescence donor molecule is attached to the surface of the particle, wherein each of the NIR dyes are disposed within, attached to, or a combination thereof, the surface of the particle.

In an embodiment, a method of detecting a target includes: providing a BRET-FRET NIR conjugate, wherein the BRET-FRET NIR conjugate includes a particle that includes a bioluminescence donor molecule and a NIR dye, wherein the particle includes a semiconductor polymer as part of the particle matrix, wherein the bioluminescence donor molecule is attached to the surface of the particle, wherein each of the NIR dyes are disposed within, attached to, or a combination thereof, the surface of the particle; introducing the BRET-FRET NIR conjugate to a host; introducing a bioluminescence initiating compound to the host; and determining the presence and location of the target corresponding to the agent by detecting the BRET-FRET NIR conjugate upon interaction with the bioluminescence initiating compound.

In an embodiment, a method of detecting a target includes: providing a BRET-FRET NIR conjugate, wherein the BRET-FRET NIR conjugate includes a particle that includes a bioluminescence donor molecule and a NIR dye, wherein the particle includes a semiconductor polymer as part of the particle matrix, wherein the bioluminescence donor molecule is attached to the surface of the particle, wherein each of the NIR dyes are disposed within, attached to, or a combination thereof, the surface of the particle; introducing the BRET-FRET NIR conjugate to a system; introducing a bioluminescence initiating compound to the system; and determining the presence of the target corresponding to the agent by detecting the BRET-FRET NIR conjugate upon interaction with the bioluminescence initiating compound.

Other systems, methods, features, and advantages will be, or become, apparent to one with skill in the art upon examination of the following drawings and detailed description. It is intended that all such additional structures, systems, methods, features, and advantages be included within this description, be within the scope of the present disclosure, and be protected by the accompanying claims.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates a schematic of self-luminescing BRET-FRET near infrared (NIR) polymer nanoparticles. The biochemical energy generated from the Luc8-catalyzed oxidation of coelenterazine is transferred initially to the MEH-PPV polymer and is then relayed to doped NIR775 dye to produce NIR emission. An amphiphilic polymer, PS-PEG-COOH, coats the nanoparticle to improve water solubility and biocompatibility. Tumor targeting ligands such as cyclic RGD peptides are conjugated to the nanoparticle surface for in vivo cancer imaging.

FIG. 2 illustrates in vitro characterization of RET IR nanoparticles. FIG. 2(a) illustrates UV-Vis absorption and fluorescence emission spectra of RET₂IR NPs in PBS buffer. FIG. 2(b) illustrates bioluminescence emission spectrum of RET₂IR NPs in PBS buffer. FIG. 2(c) illustrates representative transmission electron microscopy (TEM) image of RET₂IR NPs. Scale bar=200 nm. FIG. 2(d) illustrates dynamic light scattering (DLS) measurement of four indicated NP formulations in water by lognormal size distribution. FIG. 2(e) illustrates gel electrophoresis (0.5% agarose) analysis of RET NPs in tris-borate-EDTA (TBE) buffer: RET₁IR (lane 1), RET₁IR@cRGD (lane 2), RET₂IR (lane 3), RET₂IR@cRGD (lane 4). FIG. 2(f) illustrates bioluminescent and NIR fluorescent intensity of RET₂IR NPs (1 μg) in mouse serum at 37° C. from 0 h to 24 h. Data points represent mean±s.d. (n=3). FIG. 2(g) illustrates the NIR fluorescence signals of blood samples of mice injected with RET₁IR NPs (˜20 μg) from 5 min to 24 h. Data represent mean±s.d. (n=4). FIG. 2(h) illustrates the viability values (%) of U87MG cells estimated by MTT assay versus incubation concentrations of RET₁IR NPs. Data represent mean±s.d. (n=3).

FIG. 3 illustrates fluorescence and bioluminescence imaging of lymph nodes in mice. FIG. 3(a) illustrates fluorescence imaging of a mouse following sacrifice and necropsy 24 h after the tail-vein injection of RET₁IR NPs (˜20 μg). Superficial skin was removed before imaging but peritoneum was left intact. Autofluorescence is coded in green and NPs signal in red; NL, neck lymph nodes; AX, axillary lymph node; LT, lateral thoracic lymph node; IN, inguinal lymph node; L, left; R, right. FIG. 3(b) illustrates fluorescence image of lymph nodes excised from the mouse in FIG. 3(a): 1-4, NL; 5, AX (Left); 6, LT (Left); 7, LT (Right); 8, AX (Right); 9, IN (Left), 10 IN (Right). FIG. 3(c) illustrates bioluminescence and FIG. 3(d) illustrates fluorescence imaging of lymphatic basins in a mouse 10 mins after the injection of RET₂IR NPs (˜2 μg) intradermally in the forepaws. FIG. 3(e) illustrates bioluminescence imaging of lymphatic basins in a mouse with injection of RET₂IR NPs (˜2 μg) intradermally in the forepaws. All bioluminescence images were acquired with 10 s exposure time; PO, popliteal lymph node; LU, lumbar lymph node; L, left; R, right.

FIG. 4 illustrates fluorescence imaging of U87MG cells in vitro and in vivo with RET₁IR@cRGD NPs. (FIG. 4a-c) Live imaging of U87MG cells in vitro incubated with RET₁IR@cRGD NPs (4 μg) for FIG. 4(a) 2.5 h and FIG. 4(b) 24 h, or (c) incubated with RET₁IR NPs without cRGD for 24 h. Scale bar: 20 μm; excitation: 480/30 nm, dichroic beamsplitter: Q570LP, emission: D755/40M; objective: 20×; acquisition time: 1 s. FIG. 4(d, e) illustrates time-dependent fluorescence imaging of U87MG tumor-bearing mouse (tumors are indicated by white arrows and circles, and are 4 mm in diameter) injected with FIG. 4(d) RET₁IR@cRGD or FIG. 4(e) RET₁IR NPs (each at ˜50 μg) after 5 min, 2 h, 24 h and 48 h. Autofluorescence is coded in green and the unmixed polymer nanoparticle signal in red. FIG. 4(f) Fluorescence spectra of tissue autofluorescence (green) and the unmixed nanoparticle signal (red) in a living mouse. FIG. 4(g) ROI analysis of fluorescence intensity of tumor over background of mice in FIG. 4(d) and FIG. 4(e). Using one-tailed paired Student's t-test (n=3 mice injected with RET₁IR@cRGD), p<0.05 at 24 h. FIG. 4(h, i) NIR fluorescence imaging of urine samples FIG. 4(h) collected 48 h after injection from mouse in FIG. 4(d), and FIG. 4(i) from a mouse without any nanoparticle injection.

FIG. 5 illustrates in vivo imaging of U87MG tumors in mice with RET₂IR@cRGD. FIG. 5(a) illustrates time-dependent in vivo bioluminescence and FIG. 5(b) illustrates time-dependent fluorescence imaging of U87MG tumor-bearing mouse (indicated by a red arrow and circle; tumor size was about 5 mm in diameter) injected with RET₂IR@cRGD NPs (˜50 μg). Acquisition time for images in FIG. 5(a) from left to right: 15 s (5 min), 15 s (2 h), 1 min (24 h), 1 min (48 h), and 3 min (48 h). FIG. 5(c) illustrates ROI analysis of the bioluminescence and fluorescence intensity between tumor and background of mice in FIG. 5(a) and FIG. 5(b). Using one-tailed paired Student's t-test (n=3), p<0.00002 at 5 min, and 2 h, p<0.04 at 24 h, and p>0.05 at 48 h. FIG. 5(d) illustrates in vivo bioluminescence imaging of a mouse with a small tumor of 2 mm in diameter, as indicated by a red arrow, 2 h after tail vein injection of RET₂IR@cRGD. FIG. 5(e) illustrates NIR fluorescence imaging of urine samples collected 48 h after injection from mice in (a). Data points represent mean±s.d. (n=4). FIG. 5(f-i) illustrates histological imaging of frozen U87MG tumor slices from mouse in FIG. 5(a): FIG. 5(f) bright field, FIG. 5(g) NIR fluorescence (excitation filter: 480/30 nm, dichroic beamsplitter: Q570LP, emission filter: D755/40M, acquisition: 1 s), FIG. 5(h) Alexa Fluor 488 anti-mouse CD31 (excitation: 480/30 nm, dichroic beamsplitter: 505DCLP, emission: D535/40 nm, acquisition time: 200 ms), and FIG. 5(i) overlay of images in FIG. 5(g) and FIG. 5(h). Scale bar: 20 μm, objective: 20×.

FIG. 6 illustrates UV-Vis absorption (left) and fluorescence (right) spectra of NIR nanoparticles with indicated amounts of NIR775 dyes. The fluorescence emission of MEH-PPV decreases as the amount of NIR775 increases from 0 μg to 11 μg (MEH-PPV: 250 μg, PS-PEG-COOH: 250 μg). The intensity of NIR emission is also modulated by the amount of doped NIR775. High concentrations of NIR775 into the MEH-PPV nanoparticles matrix resulted in a decrease in the NIR fluorescence because of self-quenching of the NIR dyes. The optimal concentration range for NIR775 dye is 0.004-0.02 (NIR775/MEH-PPV in weight).

FIG. 7 illustrates the determination of the concentration of Luc8 protein using the Bradford assay (left). Data represent mean±s.d. (n=3). The absorbance at 595 nm from the RET₂IR-decomposed solution corresponds to a concentration of 16 pmol (right). The concentration of the RET₂IR NPs was calculated to be 3 pmol (A=εbC, ε₅₉₃=3,825,000^{a}). The ratio of Luc8 to the polymer nanoparticles was 5.3 (16/3)³⁷.

FIG. 8 illustrates transmission electron microscopy (TEM) images of the RET nanoparticles. (a) RET₁IR; (b) RET₁IR@cRGD; (c) RET₂IR; (d) RET₂IR@cRGD. All scale bars=500 nm.

FIG. 9 illustrates the temperature stability of RET₂IR NPs in mouse serum. Bioluminescent intensity (left) and fluorescent intensity (right) measurements were done using the same sample containing 1 μg of NPs. Data represent the mean±s.d. (n=3).

FIG. 10 illustrates the decay of signal intensity from Luc8, QDLuc8, and RET₂IR with increasing depth tissue depth was quantified in tissue-like phantom gels. (a) Representative image of a depth experiment with probe solutions in wells of a 96-well microplate. Images were acquired without gel phantom overlay (pre), and with the overlay of gel phantoms of increasing thickness ranging from 0.5 cm to 2.0 cm. The lack of Luc8 luminescence at 2.0 cm gel depth is shown at far right. (b) Luminescence intensity for each probe was quantified at each gel depth, and the plot of luminescence versus gel depth is shown. The luminescence intensity of RET₂IR (green triangles) is significantly greater than that of QDLuc8 (red squares), and both are greater than Luc8 (blue diamonds). Data represent the mean±s.d. (n=4). * Significantly difference in luminescence over range of gel depths tested (p<0.05).

FIG. 11 illustrates the luminescence intensity of Luc8, QDLuc8, and RET₂IR was compared in vivo through subcutaneous insertion of probe solution in a live nude mouse. (a) Probe solutions of Luc8 (left), QDLuc8 (center), or RET2IR (right) were prepared in a small glass tube that was inserted subcutaneously prior to image acquisition. (b) Luminescence was quantified relative to the signal obtained from prior to insertion into the mouse. Data represent mean±s.d. (n=4). (c) The acquisition of luminescence intensity of Luc8 (left), QDLuc8 (center), or RET2IR (right) through the whole animal was achieved by positioning the animal with its dorsal surface towards the camera and inserting the probe solution subcutaneously on the ventral surface of the animal. (d) Luminescence was quantified relative to the signal obtained from prior to insertion into the mouse. Data represent mean±s.d. (n=4).

FIG. 12 illustrates the change luminescence was followed over time for Luc8 (blue diamonds), QDLuc8 (red squares) and RET₂IR (green triangles) (a) in vitro, and in vivo with (b) subcutaneous and (c) whole animal probe depth penetration. All data points represent mean±s.d. (n=4). * Significantly different luminescent signal decay over time (p<0.05). † Significantly faster luminescent signal decay over time relative to all other groups (p<0.05).

FIG. 13 illustrates the fluorescence imaging of the same mouse in FIG. 3a after the removal of internal organs. Both renal and iliac lymph nodes are clearly visualized. This is a representative of a total of 6 mice imaged.

FIG. 14 illustrates bioluminescence and fluorescence imaging of the same mouse in FIG. 3e 10 days after intradermal injection of RET₂IR NPs (2 μg) at four paws. (a) Bioluminescence imaging. (b) Fluorescence imaging; Superficial skin was removed before imaging but peritoneum intact. (c) Fluorescence imaging with the surgically opened mouse and excised axillary (AX) and popliteal (PO) lymph nodes. This is a representative of a total of 3 mice imaged.

FIG. 15 illustrates live imaging of U87MG cells in vitro incubated with RET₁IR@cRGD NPs (4 μg) for 8 h. Left: bright field; middle: NIR fluorescence; right: overlay of NIR fluorescence and DAPI. The lack of overlapping between the NIR and DAPI blue fluorescence showed that the NPs were inside cells but not localized in the cell nucleus after 8 h incubation. Scale bar: 20 μm.

FIG. 16 illustrates time-dependent in vivo fluorescence imaging of a U87MG tumor-bearing mouse (indicated by white arrow; tumor size at 0.8 cm in average diameter) injected with RET₁IR@cRGD NPs (˜50 μg). Autofluorescence is coded in green and the unmixed NP signal in red. (a) 5 min, (b) 20 min, (c) 40 min, (d) 1 h, (e) 2 h, (f) 4 h, (g) 6 h, (h) 24 h, (i) 48 h. This is a representative of a total of 3 mice imaged.

FIG. 17 illustrates fluorescence images of excised mouse organs and lymph nodes after 48 h injection of RET₁IR@cRGD NPs. (a) Organs: 1 brain, 2 tumor, 3 spleen, 4 pancreas, 5 kidney, 6 lung, 7 heart, 8 bone, 9 liver, 10 muscle, 11 skin, 12 stomach. (b) Lymph nodes: AX, axillary lymph node; IN, inguinal lymph node; L, left side; R, right side of the mouse body. This is a representative of a total of 3 mice imaged.

FIG. 18 illustrates in vivo bioluminescence (a, c) and fluorescence (b, d) imaging of U87MG tumors in mice. Tumors are marked by a red arrow and circle. (a, b) Mouse with a tumor of ˜5 mm in diameter was tail-vein co-injected with RET₂IR@cRGD (˜20 μg) and free cRGD (300 μg). This is a representative of a total of n=3 mice imaged. (c, d) Mouse was tail-vein injected with RET₂IR@cRGD (˜20 μg); tumor size is ˜3 mm in diameter. The bioluminescence acquisition time was 15 s for the 5 min images and 60 s for the 2 h images. This is a representative of a total of 3 mice imaged.

FIG. 19 illustrates in vivo bioluminescence and fluorescence imaging of small U87MG tumors in mice with RET₂IR@cRGD. Tumors are marked by a red arrow and circle, and tumor size is 2-3 mm in diameter. Each mouse was tail-vein injected RET₂IR@cRGD NPs (˜50 μg). The bioluminescence acquisition time from left to right: 15 s (5 min), 15 s (2 h), and 1 min (18 h). This is a representative of a total of 3 mice imaged.

FIG. 20 illustrates fluorescence imaging of excised mouse organs and lymph nodes 48 h (a, b) or one week (c) after injection of RET₂IR@cRGD NPs. Organs in (a) and (c): 1 brain, 2 bone, 3 muscle, 4 skin, 5 kidneys, 6 lung, 7 heart, 8 spleen, 9 pancreas, 10 stomach, 11 liver, 12 tumor. (b) Lymph nodes: AX, axillary lymph nodes; IN, inguinal lymph nodes; L, left; R, right side of the mouse body. This is a representative of a total of 3 mice imaged.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of fluidics, fabrication, chemistry, biology, and the like, which are within the skill of the art. Such techniques are explained fully in the literature.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

“Bioluminescent donor protein” refers to a protein capable of acting on a bioluminescent initiator molecule substrate to generate bioluminescence.

“Bioluminescent initiator molecule” is a molecule that can react with a bioluminescent donor protein to generate bioluminescence.

Bioluminescence (BL) is defined as emission of light by living organisms that is well visible in the dark and affects visual behavior of animals (See e.g., Harvey, E. N. (1952). Bioluminescence. New York: Academic Press; Hastings, J. W. (1995). Bioluminescence. In: Cell Physiology (ed. by N. Speralakis). pp. 651-681. New York: Academic Press.; Wilson, T. and Hastings, J. W. (1998). Bioluminescence. Annu Rev Cell Dev Biol 14, 197-230.). Bioluminescence does not include so-called ultra-weak light emission, which can be detected in virtually all living structures using sensitive luminometric equipment (Murphy, M. E. and Sies, H. (1990), Meth. Enzymol. 186, 595-610; Radotic, K, Radenovic, C, Jeremic, M. (1998), Gen Physiol Biophys 17, 289-308). Bioluminescence also does not include weak light emissions, which most probably do not play any ecological role, such as the glowing of bamboo growth cone (Totsune, H., Nakano, M., Inaba, H. (1993), Biochem. Biophys. Res Comm. 194, 1025-1029). Bioluminescence also does not include emission of light during fertilization of animal eggs (Klebanoff, S. J., Froeder, C. A., Eddy, E. M., Shapiro, B. M. (1979), J. Exp. Med. 149, 938-953; Schomer, B. and Epel, D. (1998), Dev Biol 203, 1-11). Each of the citations referenced above are incorporated herein by reference.

Resonance Energy Transfer (RET) technology involves the non-radiative transfer of energy between the donor and acceptor molecule. Fluorescence Resonance Energy Transfer (FRET) and Bioluminescence Resonance Energy Transfer (BRET) technology involves the non-radiative transfer of energy between the donor and acceptor molecules by the FÖRSTER mechanism, which are well adapted for studying protein-protein interactions and protein dimerizations, but generally reserved for such measurements from cell lysates or intact cells using a microplate reader. BRET/FRET can elucidate information on real time kinetics of the interacting partners. FRET involves fluorescent molecules each as donor and acceptor molecules, allowing sensitive detection and microscopic visualization of protein interactions and intracellular signaling events in living cells. BRET technology involves a bioluminescent and a fluorescent molecule as an energy donor and acceptor respectively. The donor molecule in BRET produces light via bioluminescence.

The term “polymer” means any compound that is made up of two or more monomeric units covalently bonded to each other, where the monomeric units may be the same or different, such that the polymer may be a homopolymer or a heteropolymer. These can include biological polymers and non-biological polymers.

The term “polypeptides” includes proteins and fragments thereof. Polypeptides are disclosed herein as amino acid residue sequences. Those sequences are written left to right in the direction from the amino to the carboxy terminus In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). In addition, the protein can include non-standard and/or non-naturally occurring amino acids, as well as other amino acids that may be found in phosphorylated proteins in organisms such as, but not limited to, animals, plants, insects, protists, fungi, bacteria, algae, single-cell organisms, and the like. The non-standard amino acids include, but are not limited to, selenocysteine, pyrrolysine, gamma-aminobutyric acid, carnitine, ornithine, citrulline, homocysteine, hydroxyproline, hydroxylysine, sarcosine, and the like. The non-naturally occurring amino acids include, but are not limited to, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, S— and 4-methylproline, 3,3-dimethylproline, tert-leucine, norvaline, 2-azaphenylalanine, 3-azaphenylalanine, 4-azaphenylalanine, and 4-fluorophenylalanine.

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide includes conservatively modified variants. A substituted or inserted amino acid residue may or may not be one encoded by the genetic code. A variant of a polypeptide may be naturally occurring, such as an allelic variant, or it may be a variant that is not known to occur naturally.

Modifications and changes can be made in the structure of the polypeptides of this disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptides as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. Polynucleotide encompasses the terms “nucleic acid,” “nucleic acid sequence,” or “oligonucleotide” as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alias.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

The term “codon” means a specific triplet of mononucleotides in the DNA chain. Codons correspond to specific amino acids (as defined by the transfer RNAs) or to start and stop of translation by the ribosome.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

By “administration” is meant introducing a probe (also referred to as the “imaging agent”) of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

In accordance with the present disclosure, “a detectably effective amount” of the probe of the present disclosure is defined as an amount sufficient to yield an acceptable image using equipment that is available for clinical use. A detectably effective amount of the probe of the present disclosure may be administered in more than one injection. The detectably effective amount of the probe of the present disclosure can vary according to factors such as the degree of susceptibility of the individual, the age, sex, and weight of the individual, idiosyncratic responses of the individual, and the like. Detectably effective amounts of the probe of the present disclosure can also vary according to instrument and film-related factors. Optimization of such factors is well within the level of skill in the art.

As used herein, the term “host” or “subject” includes humans, mammals (e.g., cats, dogs, horses, etc.). Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to host or organisms noted above that are alive and are not dead. The term “living host” refers to the entire host or organism and not just a part excised (e.g., a liver or other organ) from the living host.

The term “sample” can refer to a tissue sample, cell sample, a fluid sample, and the like. The sample may be taken from a host. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue. In the present disclosure, the source of the sample is not critical.

The term “detectable” refers to the ability to detect a signal over the background signal.

The term “detectable signal” is a signal derived from non-invasive imaging techniques such as, but not limited to, near infrared detection system, bioluminescence detection system, fluorescence detection system, and the like. The detectable signal is detectable and distinguishable from other background signals that may be generated from the subject. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background.

### General Discussion

Embodiments of the present disclosure include conjugate systems, methods of using conjugate systems, RET₂IR conjugates (also referred to as “BRET-FRET-NIR conjugates”), systems including RET₂IR conjugates, methods of using the RET₂IR conjugates, and the like. In general, embodiments of the present disclosure involve the non-radiative transfer of energy between a bioluminescence donor molecule and a semiconductor polymer and then the non-radiative transfer of energy between the semiconductor polymer and an NIR dye, all without external illumination. In general, the bioluminescence donor molecule interacts with a bioluminescence initiating molecule to produce an emission. The non-radiative transfer of energy from the bioluminescence donor molecule to the semiconductor polymer causes the semiconductor polymer to emit radiation at a different wavelength than that can be transferred in a non-radiative manner to the NIR dye, which can produce NIR energy that can be detected and measured using an appropriate detection system. In this regard, embodiments of the present disclosure do not need an external light source to produce a detectable emission from the NIR dye.

As noted above, an embodiment, of the RET₂IR conjugate includes a particle (e.g., about 3 to 100 nm in diameter) that includes a bioluminescence donor molecule and a NIR dye. In an embodiment, the particle includes a semiconductor polymer as part of the particle matrix that forms the particle. In an embodiment, the bioluminescence donor molecule is attached to the surface of the particle (e.g., via a charged group such as amine group or carboxylate group on the surface of the particle). In an embodiment, the NIR dye is disposed within and/or attached to the surface of the particle.

In an embodiment, the RET₂IR conjugate can include a functional layer around the RET₂IR conjugate. In an embodiment, the functional layer is an ionic surfactant such as a cationic surfactant or an anionic surfactant. In an embodiment, the functional layer can include groups such as an amine group and/or a carboxylate group. In an embodiment, the functional layer is not a nonionic surfactant. In an embodiment, the RET₂IR conjugate does not include a functional layer around the RET₂IR conjugate.

In an embodiment, the bioluminescence donor molecule and the bioluminescence initiating compound interact to produce a bioluminescent energy. The bioluminescent energy is absorbed by the semiconductor polymer. The semiconductor polymer produces fluorescent energy in response to the non-radiative transfer of the bioluminescent energy from the bioluminescence donor molecule to the semiconductor polymer. The NIR dye absorbs the fluorescent energy and emits NIR energy in response to the energy transfer of the fluorescent energy from the semiconductor polymer to the NIR dye.

The conjugate (e.g., BRET-FRET-NIR conjugate) can be used to produce an image based on the NIR energy. In addition, multiplexed imaging of one or more targets can be performed by using a plurality of BRET-FRET NIR conjugates where each conjugate includes a NIR dye with distinct emission spectra. Additionally the conjugate (e.g., BRET-FRET-NIR conjugate) may be employed as an antibody label in immunoassays or any other in vitro or ex vivo technique measuring biological parameters.

It should also be noted that since the BRET-FRET-NIR conjugate does not need an external illumination source, the sensitivity is increased because the background signal-to-noise ratio increases. It should also be noted that the endogenous chromophores in the imaged tissue do not emit radiation without an external illumination source, where such radiation would decrease the signal-to-noise ratio.

In addition, the BRET-FRET-NIR conjugates are distinguishable and can be individually detected. In this regard, the BRET-FRET-NIR conjugates can be modified so that the BRET-FRET-NIR conjugates interact with certain targets or target compounds (e.g., chemical and/or biological compounds or polymers such a biomolecules, proteins, DNA, RNA, and the like), which allows detection of the target molecules (e.g., in vivo, ex vivo, or in cell based assays) thereby determining the area in which the target molecules are located, for example. In an embodiment, the target can include, but is not limited to, a compound, a polypeptide, a polynucleotide, an antibody, an antigen, a hapten, a cell type, a tissue type, an agent (as described herein (and for differentiation purposes only, “first”, “second”, and the like, modifiers can be added to distinguish one agent from another)), and the like.

Embodiments of the disclosure can be used in applications such as the following: cellular studies, in vivo cell trafficking, stem cell studies, tumor imaging, biomolecule array systems, biosensing, biolabeling, gene expression studies, protein studies, medical diagnostics, diagnostic libraries, microfluidic systems, delivery vehicles, multiplex imaging of multiple events substantially simultaneously, and high throughput assays for drug screening. For example, the BRET-FRET-NIR conjugates in combination with spectral imaging can be used for multiplexed imaging and detection (in vitro or in vivo) of polynucleotides, polypeptides, and the like, in a system, a host or single living cells. The BRET-FRET-NIR conjugates can be used to detect (and visualize) and quantitate events in a system, a host or a cell in in vitro as well as in in vivo studies, which decreases time and expenses since the same system can be used for cells and living organisms. For example, a drug being tested in cell culture with the BRET-FRET-NIR conjugates can then also be tested in living subjects using the same BRET-FRET-NIR conjugates.

Embodiments of the disclosure can be used to non-invasively measure selected events or interactions, the presence or absence of an agent (e.g., chemical and/or biological compounds or polymers), and the like, at a depth in an animal from about less than 20 centimeters (cm), less than about 5 cm, less than about 4 cm, less than about 3 cm, less than about 2 cm, and less than about 1 cm. For example, the BRET-FRET-NIR conjugates can be used to measure cellular events in deep tissue.

In particular, the BRET-FRET-NIR conjugates can be used in in-vivo diagnostic and/or therapeutic applications such as, but not limited to, targeting diseases and/or conditions and/or imaging diseases and/or conditions. For example, one or more embodiments of the BRET-FRET-NIR conjugates can be used to identify the type of disease, locate the proximal locations of the disease, and/or deliver agents (e.g., drugs) to the diseased cells (e.g., cancer cells, tumors, and the like) in living animals.

As mentioned above, the non-radiative energy transfer from the bioluminescence donor molecule to the semiconductor polymer or the semiconductor to the NIR dye can occur when there is an overlap (e.g., greater than 0.1%) between the emission and excitation spectra of the donor and acceptor molecules, respectively. It should be noted that the greater the overlap, the greater the efficiency. The bioluminescence energy, the fluorescence energy, and/or the NIR energy can be detected and quantified in real time using an appropriate detection system (e.g., a photomultiplier tube in a fluorometer and/or a luminometer, NIR detector, for example).

In an illustrative embodiment, the detection system used to measure the signal from the BRET-FRET-NIR conjugate includes, but is not limited to, a light tight module and an imaging device disposed in the light tight module. The imaging device can include, but is not limited to, a CCD camera and a cooled CCD camera. It should be noted that other detection systems can be used to detect the bioluminescence energy, the fluorescent energy, and/or NIR energy, such as, but not limited to, a fluorometer, a luminometer, a multiple well microplate reader, NIR detector, and the like.

In an embodiment, the BRET-FRET NIR conjugates can be detected in a system (e.g., a BRET-FRET-NIR system) using a detection system having a cooled charge-coupled device (CCD) camera, for example, capable of imaging low quantum yield of visible light ranges of about 300 to 900 nm wavelength emitted from superficial and deep tissue structures of small living subjects.

BRET-FRET-NIR conjugates

As indicated above, the BRET-FRET-NIR conjugate can include a particle (e.g., about 3 to 100 nm in diameter) that includes a bioluminescence donor molecule and a NIR dye. In an embodiment, the particle includes a semiconductor polymer as part of the particle matrix that forms the particle.

In an embodiment, the bioluminescence donor molecule and/or the NIR dye are bound (e.g., associated directly or indirectly) with the particle prior to introduction to a system or host. The term “bound” can include ways in which the bioluminescence donor molecule and/or NIR dye and the particle interact with one another to form the BRET-FRET-NIR conjugate. In general, the bioluminescence donor molecule and/or the NIR dye and the particle can be bound to one another by a physical, biological, biochemical, and/or chemical association directly or indirectly by a suitable means. The term “bound” can include, but is not limited to, chemically bonded (e.g., covalently or ionically), biologically bonded, biochemically bonded, and/or otherwise associated with the particle. In an embodiment, bound can include, but is not limited to, a covalent bond, a non-covalent bond, an ionic bond, a chelated bond, as well as being bound through interactions such as, but not limited to, hydrophobic interactions, hydrophilic interactions, charge-charge interactions, i-stacking interactions, combinations thereof, and like interactions. In an embodiment, the bioluminescence donor molecule is chemically bonded (i.e., covalently bonded) to the particle.

In general, the semiconductor polymer and/or other components can be reacted with one another to form the particle. In an embodiment, the NIR dye can be included with the semiconductor polymer and/or other components to form the particle in a nanoprecipitation procedure.

In general, a plurality of bioluminescence donor molecules can be bound to the particle. In an embodiment, the number of bioluminescence donor molecules and/or NIR dyes per particle can be controlled, at least in part, by controlling the bonding and/or conjugation conditions, the surface of the particle, the type of bioluminescence donor molecule, the type of NIR dye, and the like. The number of bioluminescence donor molecules and/or NIR dyes per particle may be about 1 to 100, 1 to 75, 1 to 50, 1 to 30, 1 to 20, and 1 to 10. In an embodiment, the ratio of the bioluminescence donor molecule and the NIR dye can be about 1:50 to 1:10. In an embodiment, the more bioluminescence donor molecules and NIR dyes per particle, the higher the emission intensity. Therefore, the number of bioluminescence donor molecules and/or NIR dyes per particle can be used to control the intensity of the NIR dye emission. Additional details about the bioluminescence donor molecule and NIR dyes are described below.

In an embodiment, the BRET-FRET-NIR conjugate can also include one or more types of agents bound (e.g., associated directly or indirectly) to the particle. The BRET-FRET-NIR conjugate can include one or more agents that can be used to enhance the interaction of the BRET-FRET-NIR conjugate with the host or subject. In an embodiment, the agent can have an affinity for a target such as, but not limited to, a compound, a polypeptide, a polynucleotide, an antibody, an antigen, a hapten, a cell type, a tissue type, and the like. In an embodiment, the agent may be an antigen specific for an antibody that corresponds to a certain disease or condition. In another embodiment, the agent may be a first protein specific for another protein. In another embodiment, the agent may be a polynucleotide sequence specific for a complementary polynucleotide sequence. In another embodiment, the agent can undergo a chemical, biological, and/or physical change, where the changed agent can have an affinity for another agent or target.

In an embodiment, the agent can include, but is not limited to, polypeptides (e.g., protein such as, but not limited to, an antibody (monoclonal or polyclonal)), nucleic acids (both monomeric and oligomeric), polysaccharides, sugars, fatty acids, steroids, purines, pyrimidines, drugs (e.g., small compound drugs), ligands, or combinations thereof. In an embodiment, the agent has an affinity for functional groups, compounds, cells, tissue, and the like, associated with a disease or condition. The agent can have an affinity for one or more targets.

In another embodiment, the agent can make the BRET-FRET-NIR conjugate bio-compatible. In other words, the BRET-FRET-NIR conjugate can include a bio-compatibility compound. The bio-compatibility compound can include compounds such as, but not limited to, polyethylene glycol; polypropylene glycol 500, dextran, and derivatives thereof. The bio-compatibility compound can be attached directly or indirectly with the particle and/or part of the particle matrix.

Thus, the agent can be selected so that the BRET-FRET-NIR conjugate can be used to image and/or diagnose the presence or absence of the compounds, polypeptides, polynucleotides, antibodies, antigens, haptens, cell types, tissue types, and the like, associated with a disease or condition, or related biological activities.

In addition, the agent can also include, but is not limited to, a drug, a therapeutic agent, radiological agent, a small molecule drug, and combinations thereof, that can be used to treat the target molecule and/or the associated disease and condition of interest. The drug, therapeutic agent, and radiological agent can be selected based on the intended treatment as well as the condition and/or disease to be treated. In an embodiment, the BRET-FRET-NIR conjugate can include two or more agents used to treat a condition and/or disease.

In an embodiment, the BRET-FRET-NIR conjugate can include at least two different types of agents, one being a targeting agent that targets certain cells or compounds associated with a condition and/or disease, while the second agent is a drug used to treat the disease. In this manner, the BRET-FRET-NIR conjugate acts as a detection component, a delivery component to the cells of interest, and a delivery component for the treatment agent. The detection of the BRET-FRET-NIR conjugate can be used to ensure the delivery of the drug to its intended destination as well as the quantity of BRET-FRET-NIR conjugates delivered to the destination.

### Bioluminescence Donor Molecule

In an embodiment, the bioluminescence donor molecule can be attached directly or indirectly to the particle in a manner as described herein, for example. In an embodiment, the bioluminescence donor molecule can be disposed (e.g., bonded, such as covalently bonded) on the surface so that it can interact with the bioluminescence initiating compound. In an embodiment, the bioluminescence donor molecule is positioned within a distance from the semiconductor polymer to facilitate the non-radiative transfer of energy from the bioluminescence donor molecule to the semiconductor polymer. In an embodiment, the particle includes about 1 to 6 bioluminescence donor molecules attached to the surface.

In an embodiment, the bioluminescence donor molecule can include, but is not limited to, luciferases, Renilla Luciferase, firefly Luciferase, aqueorin, click beetle Luciferase, Gaussia Luciferase, horse radish peroxidase (i.e., the emission can be called chemiluminescence), and other bioluminescence donor molecules than can work with semiconductor polymer, and combinations thereof. In addition, the bioluminescence donor molecule can include molecules as described in PCTUS06/34601 (entitled “Luciferases And Methods For Making And Using The Same”, filed on Sep. 6, 2006), which is incorporated herein by reference in its entirety.

In an embodiment, the bioluminescence donor molecule can include, but is not limited to, a Renilla Luciferase protein (as described herein and in the example) (Rluc, SEQ ID NO:1), a mutated Renilla Luciferase protein (as described herein and in the example) (Rluc8, SEQ ID NO:2), conservatively modified variants of each, and combinations thereof. The mutated Renilla Luciferase protein can include, but is not limited to, 8 mutations in the sequence, and these include A55T, C124A, S130A, K136R, A143M, M185V, M253L, and S287L (e.g., as described herein and in the example). In addition, the mutated Renilla Luciferase protein can include conservatively modified variants of one or more of these mutations as long as the conservatively modified variant retains the characteristics of the mutated Renilla Luciferase protein.

In an embodiment, when the bioluminescence donor molecule is a mutated Renilla Luciferase protein, the bioluminescence sensitivity increase of about 20 to 60 fold or more and about 40 fold can be realized. Also in embodiments using the mutated Renilla Luciferase protein, the mutated Renilla Luciferase protein is more stable relative to other proteins.

In general, the mutated Renilla Luciferase protein is very stable. It has been shown that a C124A mutation increases the stability of RLuc. In order to further enhance the stability of RLuc, a number of mutations can be included in addition to the C124A mutation. The combination of 8 favorable mutations including C124A generated a mutant Renilla luciferase (RLuc8) that exhibited a greater than 150-fold stability improvement in murine serum when compared to native Rluc (<1 hr versus >100 hr) and increased the sensitivity of the system by about 20 to 60 fold and about 40 fold relative to native Renilla Luciferase. In addition to being more stable, RLuc8 also exhibited at least a 4-fold improvement in light output, along with red shift of about 5 nm to its emission spectrum with respect to the native Rluc. The Renilla Luciferase protein and the mutated Renilla Luciferase protein are described in more detail in the Examples and in Nature Biotechnology 2006 (See, So M-K, Xu C, Loening A M, Gambhir S S, Rao J. and PCT Application filed on Mar. 10, 2006 having PCT/US06/08632 and entitled “BIOLUMINESCENCE RESONANCE ENERGY TRANSFER (BRET) SYSTEMS AND METHODS OF USE THEREOF”, and PCT Application filed on Sep. 6, 2006 having PCT/US2006/034601 and entitled “LUCIFERASES AND METHODS FOR MAKING AND USING THE SAME”, each of which are incorporated herein by reference).

In an embodiment, the agent and/or bioluminescence donor molecule can be linked to the particle using any stable physical and/or chemical association to the particle directly or indirectly. In general, the agent and/or bioluminescence donor molecule can be linked to the particle using, but not limited to, a covalent link, a non-covalent link, an ionic link, a chelated link, as well as being linked to the particle through interactions such as, but not limited to, hydrophobic interactions, hydrophilic interactions, charge-charge interactions, π-stacking interactions, combinations thereof, and like interactions. In an embodiment a linker can be used to link the one or more of the components (e.g., the particle, the bioluminescence donor molecules agent and the like). In an embodiment, the linker can be associated with the agent and/or bioluminescence donor and/or associated with the surface of the particle.

In an embodiment, the linker can be a compound that includes one or more functional groups to attach one or more of the particle, the agent, bioluminescence donor molecule, and/or other components of the BRET-FRET-NIR conjugate. In an embodiment, the linker can include functional groups such as, but not limited to, amines, carboxylic acids, hydroxyls, thios, and combinations thereof. The linker can include compounds such as, but not limited to, DTPA, EDTA, DOPA, EGTA, NTA, and combinations thereof.

### Bioluminescence Initiating Compound

As mentioned above, the BRET-FRET-NIR conjugate is used in conjunction with a bioluminescence initiating compound to produce a radiation emission that is absorbed by the semiconductor polymer. The bioluminescence initiating compound can include, but is not limited to, coelenterazine, analogs, and functional derivatives thereof, and D-luciferin analogs, and functional derivatives thereof. Derivatives of coelenterazine include, but are not limited to, coelenterazine 400a, coelenterazine cp, coelenterazine f, coelenterazine fcp, coelenterazine h, coelenterazine hcp, coelenterazine ip, coelenterazine n, coelenterazine O, coelenterazine c, coelenterazine c, coelenterazine i, coelenterazine icp, coelenterazine 2-methyl, and deep blue coelenterazine (DBC) (described in more detail in U.S. Pat. Nos. 6,020,192; 5,968,750 and 5,874,304, which are incorporated herein by reference). In an embodiment, the bioluminescence initiating compound can be D-luciferine when the bioluminescence compound is firefly luciferase.

In general, coelenterazines are known to luminesce when acted upon by a wide variety of bioluminescent proteins, specifically luciferases. Coelenterazines disclosed in U.S. patent application Ser. No. 10/053,482, filed Nov. 2, 2001 (which is hereby incorporated by reference in its entirety), could be used as well. Coelenterazines are available from Promega Corporation, Madison, Wis. and from Molecular Probes, Inc., Eugene, Oreg. Coelenterazines may also be synthesized as described for example in Shimomura et al., Biochem. J. 261: 913-20, 1989; Inouye et al., Biochem. Biophys. Res. Comm. 233: 349-53, 1997; and Teranishi et al., Anal. Biochem. 249: 37-43, 1997, which are incorporated herein by reference.

### Semiconductor Polymer

In an embodiment, the semiconductor polymer can include polymers that can be used to form the particle matrix. In addition, the semiconductor polymer can accept BRET energy and emit fluorescent energy upon receiving the BRET energy.

In an embodiment, the semiconductor polymer can include a polymer such as: poly[2-methoxy-5-(2′-ethylhexyloxy)-p-phenylene vinylene], poly[2-methoxy-5-(2-ethylhexyloxy)-1,4-(1-cyanovinylene-1,4-phenylene)], poly[(9,9-dioctyl-2,7-divinylenefluorenylene)-alt-co-(9,10-anthracene)], poly[{9,9-dihexyl-2,7-bis(1-cyanovinylene)fluorenylene}-alt-co-{2,5-bis(N,N′-diphenylamino)-1,4-phenylene}], poly[(9,9-dioctylfluorenyl-2,7-diyl)-alt-(benzo[2,1,3]thiadiazol-4,7-diyl)], poly[2,7-(9,9-di-octyl-fluorene)-alt-4,7-bis(thiophen-2-yl)benzo-2,1,3-thiadiazole], poly[2,6-(4,4′-bis(2-ethylhexyl)dithieno[3,2-b:2′,3′-d]silole)-alt-4,7(2,1,3-benzothiadiazole)], poly[2,7-(9,9-di-octyl-fluorene)-alt-4,7-bis(thiophen-2-yl)benzo-2,1,3-thiadiazole], poly[N-9′-heptadecanyl-2,7-carbazole-alt-5,5-(4′,7′-di-2-thienyl-2′,1′,3′-benzothiadiazole)], poly[3-hexylthiophene-2,5-diyl], and poly[2,5-bis(3-dodecylthiophen-2-yl)thieno[3,2-b]thiophene], combinations of these, and the like.

In an embodiment the one or more other components can be used in conjunction or independently from the semiconductor polymer to produce the particles. In an embodiment, the other components can include biocompatibility components, amphiphilic component, hydrophobic components, hydrophilic components, and combinations thereof. In an embodiment the amphiphilic component can include polystyrene-polyethylene glycol-COOH, and the like. In an embodiment the biocompatibility component can include n-MEG, a poly(ethylene glycol) (PEG), poly(vinyl alcohol) (PVA), poly(acrylic acid) (PAA), poly(propylene furmarate-co-ethylenee glycol) (P(PF-co-EG)), polyacrylamide, polypeptides, poly-N-substituted glycine oligomers (polypeptoids), combinations thereof, and the like, while naturally derived biocompatibility agent polymers normally include hyaluronic acid (HA), alginate, chitosan, agarose, collagen, fibrin, gelatin, dextran, and any combination thereof, as well as derivatives of each of these, and the like.

### NIR Dye

In an embodiment, the NIR can include NIR dyes that are capable of as acting as a FRET acceptor and then emit NIR energy. In an embodiment, the particle can include one or more types of NIR dyes. In an embodiment, the particle can include NIR dyes within the particle and/or on the surface of the particle. In an embodiment, the particle can include 25 to 200 NIR dyes.

In an embodiment, the NIR dyes can include, but are not limited to, BODIPY® fluorophores (Molecular Probes) (e.g., 4,4-difluoro-4-bora-3a,4a-diaza-s-indacene (and derivatives thereof), which can be modified to alter the wavelength (BODIPY® substitutes for the fluorescein, rhodamine 6G, tetramethylrhodamine and Texas Red fluorophores are BODIPY® FL, BODIPY® R6G, BODIPY® TMR and BODIPY® TR, respectively)), 1H,5H,11H,15H-Xantheno[2,3,4-ij:5,6,7-i′j′]diquinolizin-18-ium, 9-[2(or 4)-(chlorosulfonyl)-4(or 2)-sulfophenyl]-2,3,6,7,12,13,16,17-octahydro-, inner salt (molecular formula: C₃₁H₂₉ClN₂O₆S₂) (and derivatives thereof) (Texas Red), Xanthylium, 3,6-diamino-9-(2-(methoxycarbonyl)phenyl, chloride (C₂₁H₁₇ClN₂O₃) (and derivatives thereof) (NIR Rhodamine dye), and cyanine dyes (and derivatives thereof), where derivatives of each can be used to modify the wavelength. In particular, the NIR dye can include, but is not limited to, NIR775, BODIPY® dye series (e.g., BODIPY® FL-X, BODIPY® R6G-X, BODIPY®TMR-X, BODIPY® TR-X, BODIPY® 630/650-X, and BODIPY® 650/665-X (Molecular Probes, Inc. Eugene, Oreg., USA)), NIR Rhodamine dyes, NIR Alexa® dyes (e.g., Alexa® Fluor 350, Alexa® Fluor 405, Alexa® Fluor 430, Alexa® Fluor 488, Alexa® Fluor 500 (Molecular Probes, Inc. Eugene, Oreg., USA)), ADS dyes (e.g., ADS775®, ADS 780®, and the like), Texas Red, or cyanine dyes (e.g., Cy5.5 Cy3, Cy5), and Li-Cor IRDye™ products.

### Methods of Use

As mentioned above, the present disclosure relates generally to methods for studying (e.g., detecting, localizing, or quantifying) cellular events, in vivo cell trafficking, stem cell studies, tumor imaging, biomolecule array systems, biosensing, biolabeling, gene expression studies, protein studies, medical diagnostics, diagnostic libraries, microfluidic systems, and delivery vehicles. The present disclosure also relates to methods for multiplex imaging of multiple events substantially simultaneously inside a subject (e.g., a host living cell, tissue, or organ, or a host living organism) using one or more BRET-FRET-NIR conjugate without the use of an external excitation source.

In short, the BRET-FRET-NIR conjugate are introduced to the subject using known techniques. The BRET-FRET-NIR conjugate can also be labeled with one or more types of agents for the particular study (e.g., agents directed to cancer imaging and/or treatment), as mentioned above. In addition, a single agent can be associated with two or more types of BRET-FRET-NIR conjugates, where the BRET-FRET-NIR conjugates include different NIR dyes.

At an appropriate time (e.g., before, after, or at the same time as the BRET-FRET-NIR conjugate), the bioluminescence initiating compound is introduced to the host living cell, tissue, or organ, or a host living organism or can be used in a in vitro for ELISAs or for cell based assays. In an embodiment, the appropriate time may include a time frame to allow unassociated BRET-FRET-NIR conjugate to be sufficiently cleared from the appropriate area, region, or tissue of interest. The bioluminescence initiating compound can react with the bioluminescence donor molecule. The reaction causes the bioluminescence donor molecule to emit bioluminescence energy. The energy transfer from the bioluminescence donor molecule to the semiconductor polymer can occur when there is an overlap between the emission and excitation spectra of the donor and acceptor molecules, respectively. The energy is accepted by the semiconductor polymer, and then the semiconductor polymer emits fluorescent energy. The energy transfer from the semiconductor polymer to the NIR dye can occur when there is an overlap between the emission and excitation spectra of the donor and acceptor molecules, respectively. The bioluminescence energy, the fluorescent energy, and/or NIR energy can be detected and quantified in real time using a detection system. The measured signal is or can be correlated to the feature being studied. In an embodiment, the detection of the bioluminescence energy, the fluorescent energy, and/or the NIR energy can be conducted after a sufficient time frame to allow unassociated BRET-FRET-NIR conjugates to be sufficiently cleared from the appropriate area, region, or tissue of interest.

In an embodiment, the BRET-FRET-NIR conjugate can be used to study, image, diagnose the presence of, and/or treat cancerous cells, precancerous cells, cancer, or tumors. It should be noted that BRET-FRET-NIR conjugate can include an agent specific for other diseases or conditions so that other diseases or conditions can be imaged, diagnosed, and/or treated using embodiments of the present disclosure. In an embodiment, other diseases and/or conditions can be studied, imaged, diagnosed, and/or treated in a manner consistent with the discussion below as it relates to cancerous cells, precancerous cells, cancer, and/or tumors.

In an embodiment, the BRET-FRET-NIR conjugate can include one or more agents that has an affinity for cancerous cells, precancerous cells, cancer, or tumors, so that upon introduction to the subject, the BRET-FRET-NIR conjugates coordinate with the cancerous cells, precancerous cells, cancer, or tumors. Upon measuring the emitted energy from the BRET-FRET-NIR conjugate, one can image and/or diagnose the presence of the cancerous cells, precancerous cells, cancer, or tumors.

In another embodiment, the BRET-FRET-NIR conjugate includes one or more agents to treat the cancerous cells, precancerous cells, cancer, or tumors. A bioluminescence initiating compound can be introduced to the subject and react with the bioluminescence donor molecule to produce an emission from the NIR dye. Thus, upon measuring the emitted energy from the BRET-FRET-NIR conjugate, one can determine if the BRET-FRET-NIR conjugate has coordinated with the cancerous cells, precancerous cells, cancer, or tumors. Embodiments of the BRET-FRET-NIR conjugate can aid in visualizing the response of the cancerous cells, precancerous cells, cancer, or tumors to the agent.

In another embodiment, the BRET-FRET-NIR conjugate can include one or more coordinating agents that have an affinity for cancerous cells, precancerous cells, cancer, or tumors as well as one or more treating agents to treat the cancerous cells, precancerous cells, cancer, or tumors. Upon measuring the emitted energy from the BRET-FRET-NIR conjugate, one can image the cancerous cells, precancerous cells, cancer, or tumors as well as determine what portions of the cancerous cells, precancerous cells, cancer, or tumors are being treated by the treatment agent.

In general, the BRET-FRET-NIR conjugate can be used in a screening tool to select agents for imaging, diagnosing, and/or treating a disease or condition. In an embodiment, the BRET-FRET-NIR conjugate can be used in a screening tool to select agents for imaging, diagnosing, and/or treating cancerous cells, precancerous cells, cancer, or tumors. The BRET-FRET-NIR conjugate can be imaged and it can be determined if each agent can be used to image, diagnose, and/or treat cancerous cells, precancerous cells, cancer, or tumors.

### Kits

This disclosure encompasses kits that include, but are not limited to, BRET-FRET-NIR conjugate (e.g., with one or more agents as described above), a bioluminescence initiating compound, and directions (written instructions for their use). The components listed above can be tailored to the particular cellular event being studied and/or treated (e.g., cancer, cancerous, or precancerous cells). The kit can further include appropriate buffers and reagents known in the art for administering various combinations of the components listed above to the host cell or host organism.

### EXAMPLES

Now having described the embodiments of the conjugates, systems, and methods of use, in general, the example describes some additional embodiments of the conjugates, systems, and methods of use. While embodiments of conjugates, systems, and methods of use are described in connection with the example and the corresponding text and figures, there is no intent to limit embodiments of the conjugates, systems, and methods of use to this description. On the contrary, the intent is to cover all alternatives, modifications, and equivalents included within the spirit and scope of embodiments of the present disclosure.

### Example 1

### Brief Introduction:

In this Example self-luminescing near-infrared (NIR)-emitting nanoparticles are described employing an energy transfer relay that integrates bioluminescence resonance energy transfer (BRET) and fluorescence resonance energy transfer (FRET), enabling in vivo NIR imaging without external light excitation. The relay in the nanoassembly uses a mutated bioluminescent protein from Renilla luciferase as BRET donor, semiconductor polymer matrix (MEH-PPV) as BRET acceptor and FRET donor, and NIR dye as FRET acceptor. Nanoparticles were 30-40 nm in diameter, contained no toxic metals, exhibited long circulation time and high serum stability, and produced strong NIR emission. Using these nanoparticles, we successfully imaged lymphatic networks and vasculature of xenografted tumors in living mice. The self-luminescing feature provided excellent tumor-to-background ratio (>100) for imaging very small tumors (2-3 mm in diameter). Our results demonstrate that these new nanoparticles are well suited to in vivo imaging applications such as lymph node mapping and cancer imaging.

### Results:

### Preparation of BRET-FRET NIR Nanoparticles:

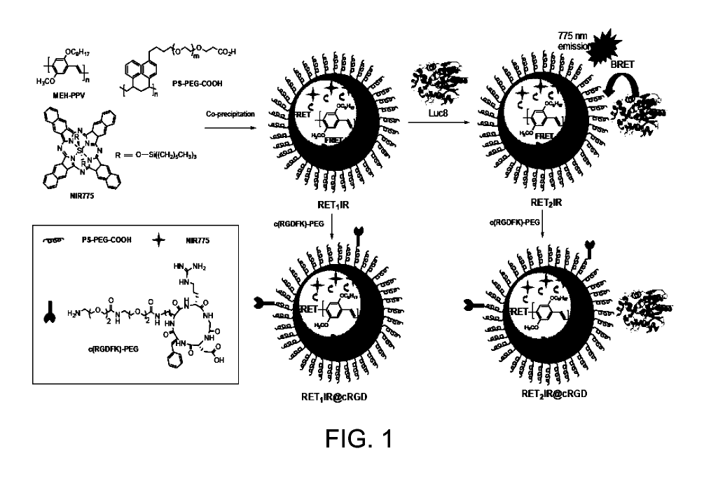

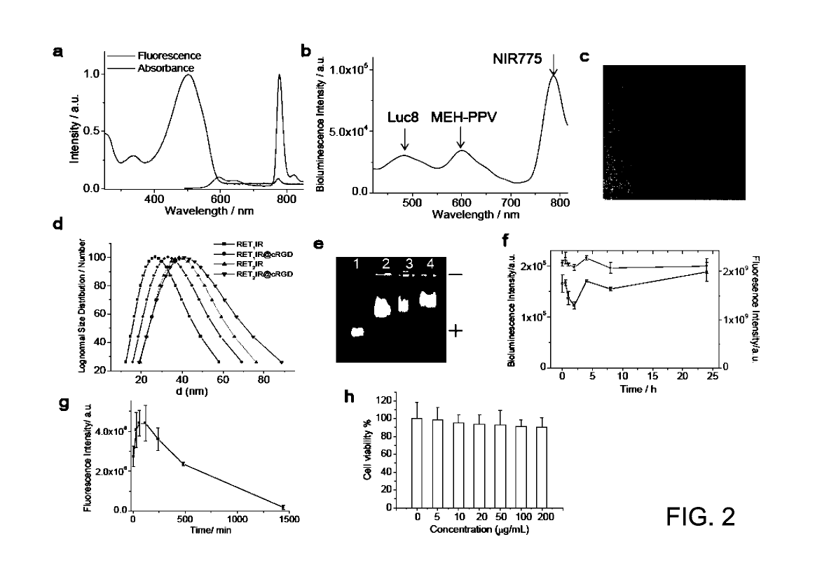

As outlined in FIG. 1, a dual resonance energy transfer relay process was used to design self-luminescing NIR nanoparticles by combining BRET and FRET. Semiconductor polymer nanoparticles with poly[2-methoxy-5-((2-ethylhexyl)oxy)-p-phenylenevinylene] (MEH-PPV) as the matrix were used because of their generally low or no toxicity, bright fluorescence intensity, and excellent photostability¹⁹⁻²⁴. MEH-PPV NPs absorb maximally at 503 nm (FIG. 2a). Luc8 (an eight-mutation variant of R. reniformis luciferase) emits blue light with a peak at 480 nm upon addition of its substrate coelenterazine²⁵. The good overlap between the MEH-PPV absorption and Luc8 emission suggests that efficient BRET can occur and generate self-luminescing polymeric nanoparticles.

To shift the luminescent emission of the nanoparticles to longer wavelengths for in vivo imaging, we doped the polymer matrix with NIR fluorescent dyes such as NIR775 (FIG. 1). Following BRET from Luc8 to the polymer matrix, FRET could occur between the polymer matrix and the doped NIR fluorophore (FIG. 2a, b). This BRET-FRET relay process produces self-luminescing BRET-FRET NIR nanoparticles (abbreviated as RET₂IR).

Polymer nanoparticles are generally hydrophobic and not water soluble, limiting their biological applications. An amphiphilic polymer, PS-PEG-COOH, was introduced to coat the nanoparticles with a biocompatible shell, orienting its hydrophobic portion within the hydrophobic core of the polymer matrix and exposing its hydrophilic groups on the nanoparticle surface (FIG. 1). This PEG coating can greatly improve the water solubility and biocompatibility of the nanoparticles, and also help reduce serum protein adsorption and suppress immune reactions, thereby increasing the blood circulation time of the nanoparticles in vivo. The carboxlyate groups presented at the PEG termini are available for bioconjugation of Luc8 and tumor-targeting ligands.

RGD (arginine-glycine-aspartic) peptides have a strong affinity for the cell adhesion receptor integrin αᵥβ₃, which plays a pivotal role in tumor angiogenesis, and have been used for in vivo imaging of a variety of cancers²⁶⁻²⁹. We conjugated cyclic RGD peptides (cRGD) via their amino groups to the PS-PEG-COOH coated RET₂IR (FIG. 1) for imaging human glioblastoma U87MG tumor xenografts in nude mice.

We first synthesized the MEH-PPV@NIR@PEG nanoparticles (abbreviated as RET₁IR since there was just FRET in this nanoparticle) using a nanoprecipitation method³⁰. To minimize the self-quenching effect among encapsulated NIR dyes, the optimal ratio of NIR775 to the MEH-PPV matrix (by weight) was found to be 0.004-0.02. Luc8 was conjugated with carbodiimide chemistry between the carboxylate groups on the nanoparticles and the free amino groups on Luc8. The number of Luc8 on each nanoparticle was estimated to be 5.3 on average. The synthesized RET₂IR NPs were stable in PBS with no aggregation observed after storage at 4° C. at a concentration of 150 μg/mL for weeks.

### Characterization of RETIR Nanoparticles:

The RET₂IR NPs exhibited a broad UV/Vis band with a maximum at 503 nm. Under excitation at 490 nm, they exhibited very weak MEH-PPV emission at 594 nm but a strong NIR peak at 778 nm (FIG. 2a). At a blending ratio of 0.012 (NIR775/MEH-PPV by weight), the fluorescence quantum yield of the RET₂IR NPs at 594 nm emission (the polymer matrix) in water dropped from its original value of 0.18 to 0.03 after doping with NIR775, while the quantum yield of the 778 nm emission (NIR775) in water was around 0.09 (in comparison, the quantum yield of free NIR775 in THF was 0.08). This result confirms that FRET occurred efficiently from the MEH-PPV matrix to NIR775 in the RET₂IR NPs.

We examined the bioluminescence emission of the RET₂IR NPs upon addition of coelenterazine. In addition to the emission of Luc8 at 480 nm, two new emission peaks at 594 nm (relatively weak, from MEH-PPV matrix) and 778 nm (strongest, from NIR775) were detected (FIG. 2b); this is consistent with our design of the BRET-FRET relay process from Luc8 to the polymer matrix and then to NIR775. The overall BRET ratio, determined by dividing the acceptor emission (550-810 nm) by the donor emission (400-550 nm), was 2.5 (corresponding to an efficiency of 71%), which was nearly double that of QDLuc8 nanoparticles that were previously reported to have a BRET ratio of 1.29⁷.

Transmission Electron Microscopy (TEM) and Dynamic Light Scattering (DLS) indicated an average particle diameter of approximately 27 nm for the RET₁IR NPs (FIG. 2c, d). Conjugation to Luc8 and cRGD increased the average hydrodynamic diameter to approximately 33 nm, 38 nm and 41 nm for the cRGD conjugated RET₁IR (RET₁IR@cRGD) NPs, RET₂IR NPs and the cRGD conjugated RET₂IR (RET₂IR@cRGD) NPs, respectively (FIG. 2d). Gel electrophoresis analysis of these nanoparticles in 0.5% agarose showed a different mobility than the original RET₁IR NPs due to changes in the surface charge and particle size following bioconjugation (FIG. 2e).

We examined the stability of the RET₂IR NPs in mouse serum by monitoring both their bioluminescence and fluorescence at 37° C. for 24 h (FIG. 2f). The bioluminescence intensity of nanoparticles remained stable over 24 h and even increased slightly by 10% at 24 h. The fluorescence intensity of the nanoparticles was nearly unchanged with just 3% decrease after 24 h. As expected, the stability of the RET₂IR NPs in mouse serum was higher at 4° C. and 22° C. than at 37° C. In comparison to QDs, the RET₂IR NPs are more stable in mouse serum and suitable for in vivo imaging applications¹⁶. The circulation time of the RET₁IR NPs in the blood was long with an estimated half-life of approximately 8 hours (FIG. 2g).

The depth of signal penetration of RET₂IR was assessed both in vitro in a tissue phantom and in vivo in live mice in direct comparison to Luc8 and QDLuc8. In the tissue phantom, RET₂IR luminescence signal was observable even at 2 cm of gel depth at signal intensities significantly greater than QDLuc8, while the signal depth penetration of Luc8 was limited to 1.5 cm. In live mice, the luminescence signal penetration subcutaneously and through the whole animal of RET₂IR was significantly greater than Luc8, and was equivalent to QDLuc8. In fact an observed advantage of RET₂IR nanoparticles was the maintained evolution of luminescence signal following the administration of coelenterazine relative to QDLuc8, which exhibited faster signal decay kinetics especially under deeper penetration depths in vivo.

The cytotoxicity of the RET₁IR NPs was evaluated by the MTT assay in human glioblastoma U87MG cells, and no significant differences in cell viability were observed in the absence or presence of the RET₁IR NPs at a concentration of 5-200 μg/mL at 37° C. for 24 h (FIG. 2h). In conjunction with the imaging depth of penetration studies, this data demonstrates that RET₂IR nanoparticles are a safer alternative to QDLuc8 as they are free from toxic heavy metals, and that there is no loss of imaging depth penetration performance in vivo.

Lymph Node Imaging with RETIR Nanoparticles:

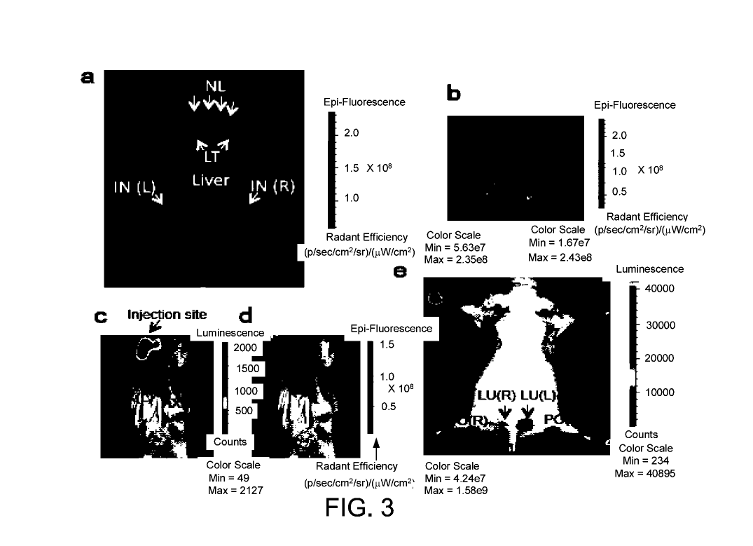

At 24 h after tail vein injection of the RET₁IR NPs into nude mice, the mice were surgically opened for imaging (FIG. 3a, b). Strong NIR fluorescence signals were detected in the lymphatic networks: neck lymph nodes (NL), axillary lymph nodes (AX), lateral thoracic lymph nodes (LT), and inguinal lymph nodes (IN) could all be clearly imaged with NIR fluorescence emission. After the removal of internal organs, renal and iliac lymph nodes were also clearly visualized with strong NIR fluorescence. The lymphatic system is difficult to identify because its channels are small and not directly accessible. Nanoparticles have been evaluated for labeling the AX, IN, or neck lymph nodes³¹⁻³⁶. Our results show that the RET₁IR NPs allow highly efficient labeling of all the lymph nodes in the lymphatic networks after tail-vein injection.

Similarly, the RET₂IR NPs (˜2 μg) were introduced into the forepaws of mice via intradermal injections. Within 10 min of injection, the AX could be readily visualized non-invasively by bioluminescence imaging (FIG. 3c). When followed by in vivo fluorescence imaging, lower fluorescent signals were obtained at the AX in comparison with bioluminescence imaging (FIG. 3d). After RET₂IR NPs were injected into all four paws (each at ˜2 μg), all lymph nodes in the same mouse were clearly visualized by bioluminescence imaging (FIG. 3e). The axillary and popliteal lymph nodes were still clearly labeled even 10 days later.

Tumor Imaging with RET₁IR Nanoparticles:

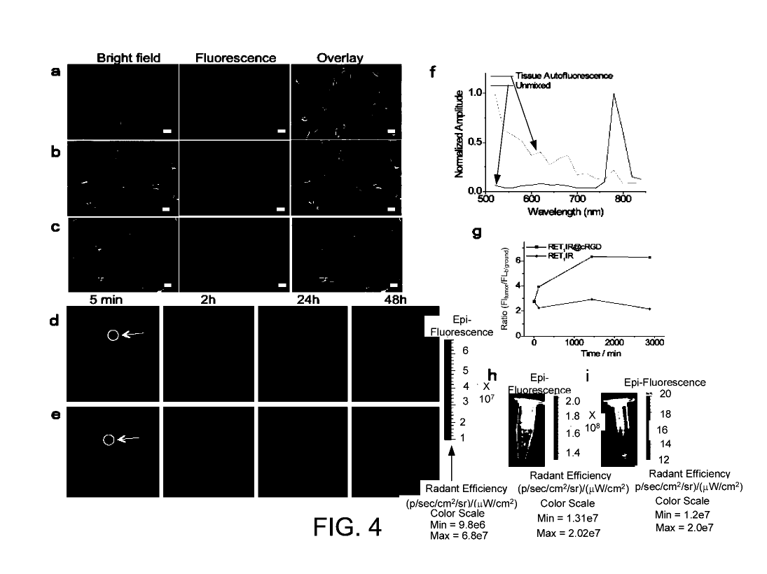

To evaluate RET₂IR NPs for cancer imaging, we first tested the cRGD conjugated RET₁IR (RET₁IR@cRGD) NPs for imaging αᵥβ₃ of human glioblastoma U87MG cells in culture, which express high levels of αᵥβ₃. After 2.5 h incubation, intense NIR fluorescence was detected at the cell membrane and in the cytoplasm (FIG. 4a), of which the cytoplasmic signal increased with time up to 24 h (FIG. 4b). DAPI staining revealed little NIR fluorescence signal in the nucleus. In contrast, the control RET₁IR NPs without cRGD resulted very weak NIR fluorescence from U87MG cells under the same experimental conditions (FIG. 4c).

The RET₁IR@cRGD NPs were then injected intravenously into nude mice bearing a U87MG tumor on the left shoulder (˜50 μg per animal), and the mice were imaged at multiple time points post injection (FIG. 4d). The in vivo fluorescence spectrum of the RET₁IR@cRGD NPs was collected from 520 nm to 840 nm with a bandwidth of 20 nm and an excitation of 465 nm (FIG. 4f). As early as 5 min post injection, enhanced NIR fluorescence signal (780 nm) was observed in the U87MG tumor (FIG. 4d), and gradually increased over time. ROI measurements showed that the NIR fluorescence signal ratio between the tumor and the surrounding tissue was 2.4±0.6 (5 min), 3.3±0.7 (2 h), 6.1±0.2 (24 h) and 5.9±0.6 (48 h) (FIG. 4g). There was significant fluorescence signal from the skin after 2 h, which complicated the detection of small size tumors, however the NIR fluorescence signal became stronger when the tumor size became larger, for example, at 8 mm in diameter. In comparison, no significant NIR fluorescence signal was observed in the tumors injected with the non-targeting RET₁IR NPs (FIG. 4e), and the NIR fluorescence signal ratio between the tumor and background was 2.7, 2.2, 2.9 and 2.1 at 5 min, 2 h, 24 h and 48 h, respectively (FIG. 4g). The small increase in the NIR fluorescence signal also observed with the non-targeting RET₁IR NPs is likely due to the enhanced permeability and retention (EPR) effect.

Fluorescence imaging of sacrificed mice 48 h after injection of RET₁IR@cRGD NPs revealed uptake in the lymph nodes, skin, stomach, bone, liver, spleen and tumor. NIR fluorescence signal was also observed in collected urine (FIG. 4h), suggesting that RET₁IR NPs were cleared from the body through the renal system in addition to the hepatobiliary system.

Tumor Imaging with RET₂IR Nanoparticles:

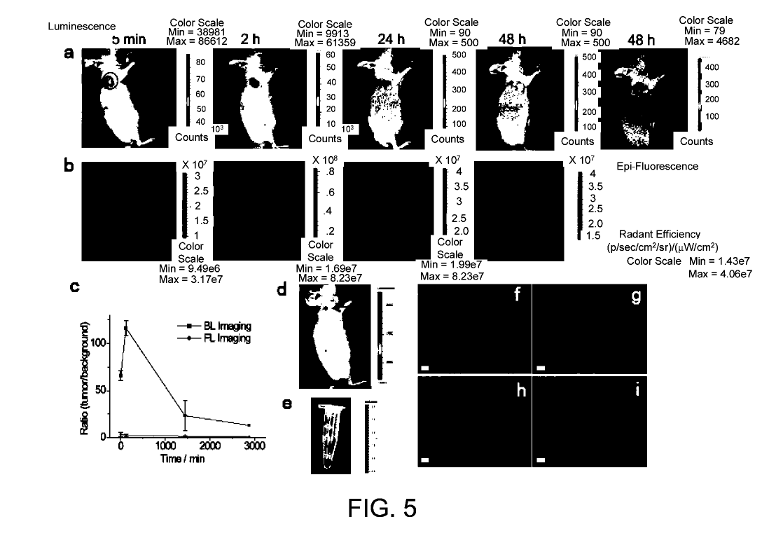

The RET₂IR NPs can emit NIR light in the presence of the substrate of Luc8, coelenterazine, without external excitation, and therefore can provide further advantages in comparison to the RET₁IR NPs. The cRGD conjugated RET₂IR@cRGD NPs were similarly evaluated in the U87MG tumor xenograft mouse model. After tail vein injection of the RET₂IR@cRGD NPs, mice were image serially by bioluminescence imaging. At 5 minutes post injection, strong bioluminescence emission was observed in the U87MG tumor with little signal from other tissues (FIG. 5a). The bioluminescence signal ratio between the tumor and background was estimated from ROI measurements to be 66.0±4.8 (5 min), 116.4±7.6 (2 h), 23.3±16.2 (24 h) and 12.9 (48 h) (FIG. 5c). The bioluminescence signal from the U87MG tumor decreased significantly when free cRGD (300 μg) was co-injected intravenously with the RET₂IR@cRGD NPs, confirming the specific binding of RET₂IR@cRGD NPs to αᵥβ₃ integrin expressed at the neovascular endothelium of the U87MG tumor.

The RET₂IR NPs allow simultaneous fluorescence and bioluminescence imaging in the same mouse, therefore fluorescence spectral imaging was conducted for comparison. Intense NIR fluorescence signal was observed in the liver, and the NIR fluorescence signal in the tumor was much weaker than the bioluminescence signal (FIG. 5b). The fluorescence signal ratio between the tumor and surrounding tissues was 3.4±2.4, 2.4±1.7, 1.3±0.6, and 0.9 at 5 min, 2 h, 24 h and 48 h, respectively (FIG. 5c). These results demonstrate that bioluminescence imaging provided much higher sensitivity than fluorescence imaging of tumors in vivo.

We further tested the efficiency of the RET₂IR NPs in imaging smaller tumors (tumor size of 2-3 mm in diameter) (FIG. 5d). A strong bioluminescence signal was still observed in the U87MG tumor of the mice at 5 min and 2 h. In comparison, the fluorescence signal in the U87MG tumor was much weaker. These results further demonstrated that bioluminescence showed higher sensitivity than fluorescence in cancer imaging in vivo.

In vivo biodistribution of the RET₂IR NPs was similar to that of the RET₁IR@cRGD NPs except for increased uptake in the lung, which is to be expected due to the relatively larger size of the RET₂IR NPs. Renal clearance also occurred with the RET₂IR NPs as the NIR fluorescence signal was observed in the urine sample collected at 48 h post injection (FIG. 5e).

Tumor tissues were excised for sectioning and immunohistochemical staining to confirm the tumor-specific uptake of the RET₂IR NPs (FIG. 5f-i). Tumor slices were fixed and stained with vascular endothelium-specific CD31 antibodies to mark tumor blood vessels for 1 h before imaging under a fluorescence microscope. Strong NIR fluorescence was observed within the tumor tissue (FIG. 5g), which overlaps well with the green fluorescence signal from the Alexa Fluor 488-conjugated CD31 antibody (FIG. 5h, i). This result suggests the binding of the RET₂IR@cRGD NPs at the neovascular endothelium of angiogenic tumor blood vessels.

### Discussion:

While QDs possess excellent optical properties as an imaging probe, concerns such as unfavorable in vivo pharmacokinetics after systemic administration, their instability in vivo and intrinsic toxicity due to their formation from toxic heavy metals have limited their use for translational research. In comparison, the self-luminescing NIR NPs show high stability in mouse serum and increased circulation time (FIG. 2f & g). Importantly, they do not contain any heavy toxic metals. At a size of around 30-40 nm in diameter, they primarily undergo hepatobiliary clearance in mice, but renal clearance has also been observed (FIG. 4h & FIG. 5e). Combined, all these features make the RET₂IR nanoparticles highly attractive for in vivo imaging research.

An important application of fluorescent nanoparticles is lymph node mapping and imaging that traditionally uses vital blue dyes and radioactive tracers in clinics³⁵. We have shown highly efficient labeling of all lymph nodes in the lymphatic networks of mice after a single tail-vein injection of RET₁IR NPs.

Bioluminescence imaging with RET₂IR NPs gave a tumor-to-background ratio of over 100 and very small subcutaneously implanted tumors (2 or 3 mm in diameter) have been clearly imaged within just 5 mins after the tail-vein injection of the RET₂IR NPs. This level of sensitivity is at least an order of magnitude higher than fluorescence imaging. With further optimization, tumors smaller than 1 mm in diameter may be readily detectable with the self-luminescing NIR probes.

To the best of our knowledge, our RET₂IR NP is the first demonstration of a BRET-FRET relay process for in vivo imaging. The relay uses a Renilla luciferase mutant as the BRET donor, semiconductor polymer MEH-PPV matrix as both the BRET acceptor and the FRET donor, and an NIR dye as the FRET acceptor. The energy transfer relay process was surprisingly efficient. The BRET-FRET relay strategy now allows the use of fluorescent materials other than QDs in building self-luminescing NIR probes for in vivo imaging as long as proper fluorophores are chosen to relay the excitation energy from Renilla luciferase to the NIR dye dopant, even in multiple relaying steps. Further fine tuning of the probe by matching multiple BRET and FRET pairs should enable self-luminescing NIR NPs for multiplex in vivo imaging²¹.

### Methods:

### Materials:

The poly(phenylene vinylene) derivative poly[2-methoxy-5-(2-ethylhexyloxy)-1,4-phenylenevinylene] (MEH-PPV; MW: 200,000 Da; polydispersity, 4.0) were purchased from ADS Dyes, Inc. (Quebec, Canada). Polystyrene (PS) and polystyrene graft ethylene oxide functionalized with carboxyl groups (PS-PEG-COOH; MW: 21,700 Da of the PS moiety; 1,200 Da of PEG-COOH; polydispersity, 1.25) were purchased from Polymer Source Inc. (Quebec, Canada). Silicon 2,3-naphthalocyanine bis(trihexylsilyloxide) (NIR775) was purchased from Sigma Aldrich, Inc. Cyclic RGD peptide was purchased from Peptides International, Inc. All the other chemicals were purchased from Sigma Aldrich, Inc. and used without purification. Luc8 was prepared according to our previously published procedures⁸.

### Synthesis of NIR Nanoparticles:

NIR775-doped NIR nanoparticles were prepared using a nanoscale precipitation technique³⁰. In a typical procedure, a solution of tetrahydrofuran (THF) containing 50 μg/mL of MEH-PPV, 50 μg/mL of PS-PEG-COOH, and 0.6 μg/mL of NIR775 dye was prepared. An aliquot of the mixture (5 mL) was then quickly dispersed into 10 mL of water under vigorous sonication. Extra THF was evaporated at an elevated temperature (below 90° C.) under the protection of nitrogen. The THF-free NPs solution was filtrated through a 0.2 μm cellulose membrane filter. Bioconjugation was carried out by the EDC-mediated coupling reaction between the carboxyl groups on the NPs and the amine-containing molecules (Luc8 and RGD peptide). In a typical conjugation reaction, 60 μL, of concentrated HEPES buffer (1 M) were added to 3 mL of a solution of the carboxylate-presenting NP (50 μg/mL in water) and the amine-containing molecules (100 μL, of Luc8 at 3 mg/mL and 30 μL, of RGD peptide at 10 mg/mL), followed by vortex mixing. Then, 50 μL, of freshly prepared aqueous EDC solution (10 mg) was added and the above mixture was magnetically stirred for 1 hour at room temperature. The uncoupled free Luc8 and excess EDC were removed by four washes using a 100 K Amicon Ultra filter (Millipore Corporation) under centrifugation at 3,000 rpm for 3 min at 4° C. The final complex was kept in PBS buffer at 4° C.

### In Vitro Nanoparticles Characterization:

The size and morphology of the nanoparticles were investigated using Transmission Electron Microscope (TEM) (FEI Tecnai G2 F20 X-TWIN, 200 kV). TEM samples were prepared by dripping the NP solution onto a carbon-supported copper grid and drying it at room temperature before observation. The hydrodynamic size of the nanoparticles was also measured in aqueous solution by Dynamic Light Scattering (DLS) (Brookhaven 90 Plus Nanoparticle Size Analyzer). The absorption spectra were recorded on an Agilent 8453 UV-V is spectrometer. Fluorescence and bioluminescence emission spectra were collected with a FluoroMax-3 (Jobin Yvon Inc.) and corrected for wavelength-dependent detector sensitivity as described by the company. In the case of bioluminescence, the excitation light was blocked.

### Cell Culture, Cytotoxicity Assay and Cell Imaging:

U87MG (human glioblastoma, high αᵥβ₃ expression) cells were grown in DMEM supplemented with 10% FBS. Cultures were maintained at 37° C. under a humidified atmosphere containing 5% CO₂. Cytotoxicity in the U87MG cell line was measured using a methyl thiazolyl tetrazolium (MTT) assay. Cells growing in log phase were seeded into a 96-well cell-culture plate at 1×10⁴ cells/well and then incubated for 24 h at 37° C. under 5% CO₂. RET₁IR NPs (100 μL/well) were added to the wells of the treatment group at varying concentrations, and 100 μL/well DMEM was added to the negative control group, followed by incubation of the cells for 24 h at 37° C. under 5% CO₂. Subsequently, 10 μL of MTT (5 mg/mL) was added to each well of the 96 well assay plate and incubated for an additional 4 h at 37° C. under 5% CO₂. After the addition of DMSO (200 μL/well), the assay plate was allowed to shake at room temperature for 20 min. A Tecan microplate reader was used to measure the OD₅₇₀ (Absorbance value) of each well with the background subtraction at 690 nm. The following formula was used to calculate the viability of cell growth:

cell viability (%)=(mean of Absorbance value of treatment group/mean of Absorbance value of control)×100.

For cell imaging experiments, 5×10⁵ cells per well were seeded on 18 mm glass coverslips and cultured for 24 h before imaging with a Zeiss Axiovert 200M Microscope (excitation: 480/30 nm; dichroic beamsplitter: Q570LP; emission: D755/40M; objective: 20×; acquisition time: 1 s).

### Blood Circulation Half-Life in Mice:

The RET₁IR NPs (˜20 μg) were injected intravenously into the tail veins of three six-week old nude mice. At various time points postinjection, ˜20 μL of blood was collected from the tail into 50 μL of 0.9% NaCl solution containing 1.5 mg/mL of EDTA. The NIR fluorescence intensity of blood samples was assayed on an IVIS spectrum imaging system (excitation: 465±15 nm; emission: 780±10 nm). Blood samples without the RET₁IR NPs were measured to determine the blood autofluorescence level, which was subtracted from the fluorescence intensity of injected samples.

### Biodistribution Studies:

Mice were euthanized by cervical dislocation under deep isoflurane anesthesia. Urine samples were immediately collected, and lymph nodes, brain, spleen, pancreas, kidney, lung, heart, liver, bone (femur), muscle (hind leg), stomach (emptied), and dorsal skin were harvested; for tumor-bearing mice, tumors were also harvested. Tissues were subjected to fluorescence imaging using an IVIS spectrum imaging system immediately (excitation: 465±15 nm; emission: 780±10 nm).

### Lymph Node Imaging:

Mice were anesthetized with 2.5% isoflurane, and RET₁IR NPs (˜20 μg) were administered to nude mice by tail-vein catheterization using the Vevo MicroMarker TVA (Vascular Access) Cannulation Kit (VisualSonics). The tail vein was further flushed with 100 μL of PBS buffer. At 24 h after injection, mice were euthanized, dissected to locate the lymph nodes of interest, and imaged using an IVIS spectrum imaging system immediately (excitation: 465±15 nm; emission: 780±10 nm). Alternatively, ˜10 μL of RET₂IR NPs (˜2 μg each) were administered to the forepaws via intradermal injections. Within 10 min of injection, mice received an intravenous injection of 10 μg of coelenterazine for in vivo bioluminescence imaging (acquisition time: 10 s; no emission filter). Following bioluminescence imaging, in vivo fluorescence imaging was carried out (excitation: 465±15 nm; emission: 780±10 nm).

### Tumor Implantation and In Vivo Imaging:

Animal procedures were approved by the Institutional Animal Care Use Committee of Stanford University. Tumor cells were harvested by incubation with 0.05% trypsin-EDTA when they reached near confluence. Cells were pelleted by centrifugation and resuspended in sterile PBS. U87MG cells (2×10⁶ cells/site) were implanted subcutaneously into the left shoulder of four- to five-week-old female nude mice (Charles River Breeding Laboratories). When the tumors reached the size of 2 to 8 mm in diameter (two to three weeks after implantation), the tumor-bearing mice were subjected to biodistribution and imaging studies. In vivo and ex vivo fluorescence imaging was performed with an IVIS spectrum imaging system (excitation: 465±15 nm filter; emission: collected from 520 nm to 840 nm with a bandwidth of 20 nm).

For bioluminescence imaging, the mice were imaged after tail vein injection of coelenterazine (20 μg/mouse in 20 μL of methanol and 80 μL of phosphate buffer). Images were acquired without filters.

### Histology:

Tumor-bearing mice were sacrificed 48 h after injection with RET₂IR@cRGD. Tumor tissues were collected, washed with PBS, frozen by dry ice and stored at −80° C. Frozen samples were cryosectioned by microtome at −20° C. into slices of 5 μm thickness, and then fixed in cold acetone for 5 min (−20° C.). Nonspecific binding sites were blocked over 30 minutes with PBS containing 10% mouse serum. The sections were stained with 1 μg of Alexa Fluor 488 anti-mouse CD31 antibody (Biolegend Inc., San Diego, Calif.) in 100 μL PBS buffer for 1 h at 37° C. The sections were washed with PBS and analyzed under a Zeiss Axiovert 200M Microscope.

### Supplementary Methods

### Depth Phantom and In Vitro BRET Kinetics

A gelatin-based phantom was formed as previously described³ for side-by-side comparison of the imaging depth penetration of Luc8, QDLuc8, and RET₂IR. QDLuc8 was made by conjugating Luc8 to QD800 (Invitrogen) according to previously published procedures³⁸. Briefly, a mixture of porcine gelatin (10% w/v), porcine hemoglobin (170 mM), and Intralipid© (1% v/v) were mixed in buffer containing 50 mM Tris HCl (pH 7.4), 150 mM NaCl, and 0.1% w/v NaN₃ at elevated temperatures. The solution was then poured at 4° C. into rectangular molds to specific gel depths of 0.5 cm and 1.0 cm and let set overnight. Probe solutions (20 pmol in 300 μL water) equilibrated to Luc8 protein mass were placed into wells of a 96-well plate and luminescence was measured using an IVIS Spectrum imager with a filter setting of ‘open’. Images were acquired without gel phantom (Pre), and with combinations of gel phantoms resulting in 0.5 cm, 1.0 cm, 1.5 cm, and 2.0 cm total gel phantom depths. Signal intensities were calculated from regions of interest and were corrected to normalize initial emission intensity between the three luminescent probe samples prior to gel overlay. The percent signal remaining was quantified over all gel depths (n=3) and the trend of signal decay was assessed for statistical significance by general linear model repeated measures analysis at a=0.05.

The kinetics of signal decay from all three probes was determined in vitro in a 96-well plate without any phantom gel overlay. Images were acquired from time 0 min to 30 min using an IVIS Spectrum imager, and luminescence intensity was quantified by region of interest measurement. The ratio of measured signal to signal at time 0 min was plotted as the % signal remaining over time (n=3), and the trend of differences in signal decay kinetics was assessed by general linear model repeated measures analysis at a=0.05.

### In Vivo Imaging Depth Measurement and BRET Kinetics

A female nude mouse was anesthetized and a horizontal 1 cm incision was made in the skin 0.5 cm rostral to the base of the tail. A 100×4 mm glass tube closed at one end was filled with 30 μL of a mixture of 10 pmol probe and 1 μg coelenterazine, and the tube was inserted 3 cm subcutaneously between the skin and peritoneum. Images were acquired using an IVIS Spectrum imager with a filter setting of ‘open’ with the mouse ventral side up (subcutaneous) and then with the mouse dorsal side up (whole animal). Luminescence intensity was quantified by region of interest and the % signal remaining relative to an image of the probe solution taken outside of the animal prior to tube insertion was calculated. Comparisons of average % signal remaining were performed using one-way ANOVA followed by Tukey's test for honestly significant differences (n=3).

The kinetics of luminescent signal decay from all three probes was determined using the method of inserting a glass tube into a mouse subcutaneously described above. Images were acquired both from the ventral surface (subcutaneously) and from the dorsal surface (whole animal) from 5 min to 25 min after addition of coelenterazine into the probe solution using an IVIS Spectrum with a filter setting of ‘open’. The % signal remaining was calculated relative to the luminescence intensity at time 5 min, as quantified by region of interest measurement, and the trend of signal decrease was assessed using a general linear model repeated measures analysis at α=0.05 (n=3).

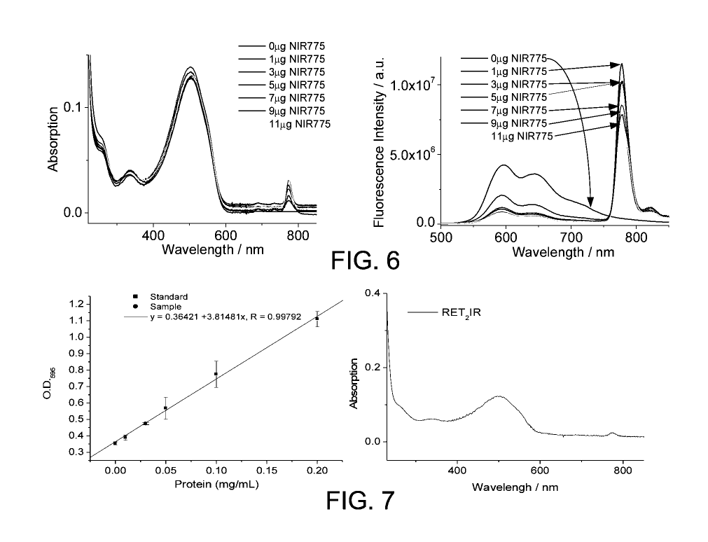

FIG. 6 illustrates UV-Vis absorption (left) and fluorescence (right) spectra of NIR nanoparticles with indicated amounts of NIR775 dyes. The fluorescence emission of MEH-PPV decreases as the amount of NIR775 increases from 0 μg to 11 μg (MEH-PPV: 250 μg, PS-PEG-COOH: 250 μg). The intensity of NIR emission is also modulated by the amount of doped NIR775. High concentrations of NIR775 into the MEH-PPV nanoparticles matrix resulted in a decrease in the NIR fluorescence because of self-quenching of the NIR dyes. The optimal concentration range for NIR775 dye is 0.004-0.02 (NIR775/MEH-PPV in weight).

FIG. 7 illustrates the determination of the concentration of Luc8 protein using the Bradford assay (left). Data represent mean±s.d. (n=3). The absorbance at 595 nm from the RET₂IR-decomposed solution corresponds to a concentration of 16 pmol (right). The concentration of the RET₂IR NPs was calculated to be 3 pmol (A=εbC, ε₅₉₃=3,825,000^{a}). The ratio of Luc8 to the polymer nanoparticles was 5.3 (16/3)³⁷.

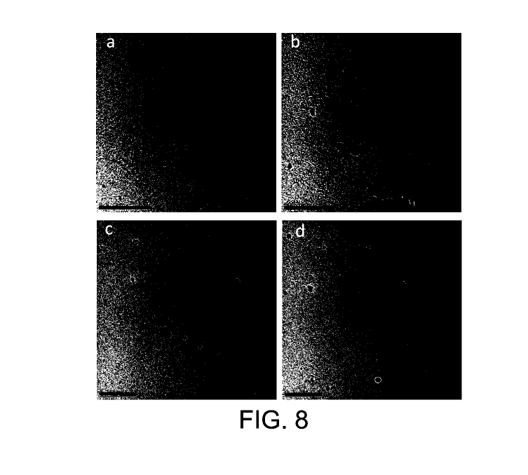

FIG. 8 illustrates transmission electron microscopy (TEM) images of the RET nanoparticles. (a) RET₁IR; (b) RET₁IR@cRGD; (c) RET₂IR; (d) RET₂IR@cRGD. All scale bars=500 nm.

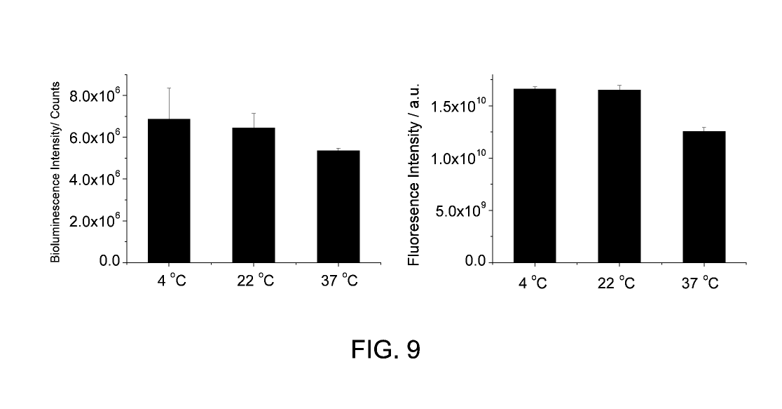

FIG. 9 illustrates the temperature stability of RET₂IR NPs in mouse serum. Bioluminescent intensity (left) and fluorescent intensity (right) measurements were done using the same sample containing 1 μg of NPs. Data represent the mean±s.d. (n=3).

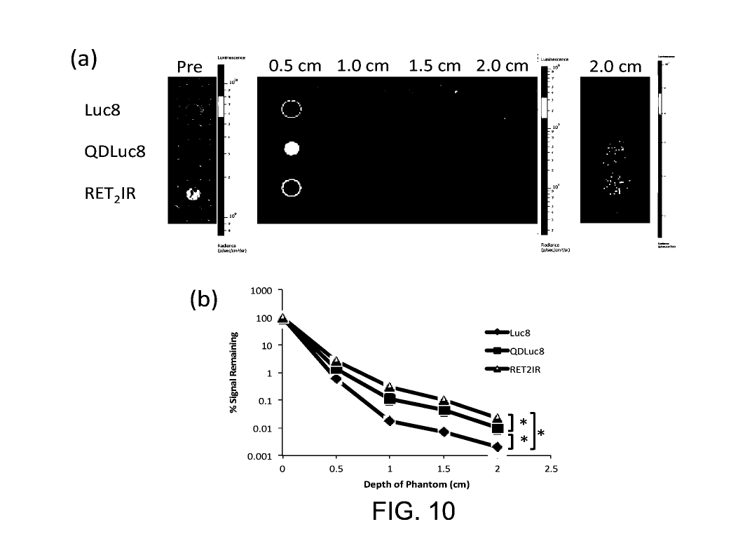

FIG. 10 illustrates the decay of signal intensity from Luc8, QDLuc8, and RET₂IR with increasing depth tissue depth was quantified in tissue-like phantom gels. (a) Representative image of a depth experiment with probe solutions in wells of a 96-well microplate. Images were acquired without gel phantom overlay (pre), and with the overlay of gel phantoms of increasing thickness ranging from 0.5 cm to 2.0 cm. The lack of Luc8 luminescence at 2.0 cm gel depth is shown at far right. (b) Luminescence intensity for each probe was quantified at each gel depth, and the plot of luminescence versus gel depth is shown. The luminescence intensity of RET₂IR (green triangles) is significantly greater than that of QDLuc8 (red squares), and both are greater than Luc8 (blue diamonds). Data represent the mean±s.d. (n=4). * Significantly difference in luminescence over range of gel depths tested (p<0.05).

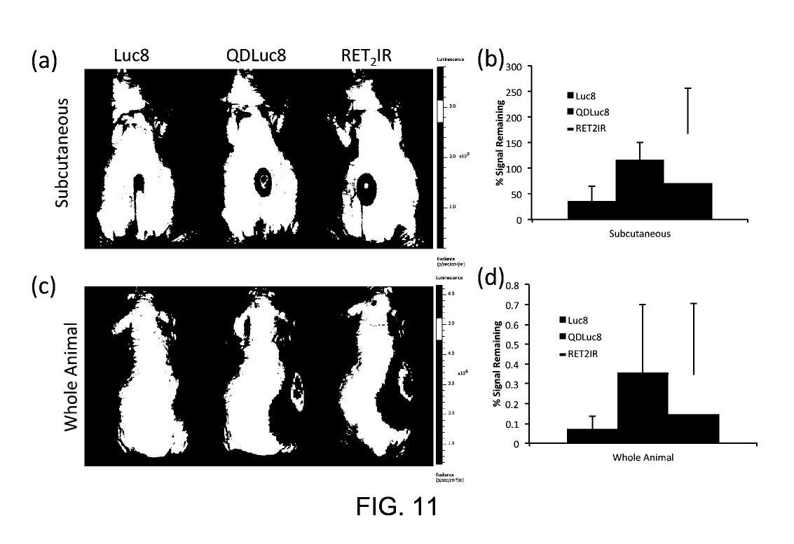

FIG. 11 illustrates the luminescence intensity of Luc8, QDLuc8, and RET₂IR was compared in vivo through subcutaneous insertion of probe solution in a live nude mouse. (a) Probe solutions of Luc8 (left), QDLuc8 (center), or RET2IR (right) were prepared in a small glass tube that was inserted subcutaneously prior to image acquisition. (b) Luminescence was quantified relative to the signal obtained from prior to insertion into the mouse. Data represent mean±s.d. (n=4). (c) The acquisition of luminescence intensity of Luc8 (left), QDLuc8 (center), or RET2IR (right) through the whole animal was achieved by positioning the animal with its dorsal surface towards the camera and inserting the probe solution subcutaneously on the ventral surface of the animal. (d) Luminescence was quantified relative to the signal obtained from prior to insertion into the mouse. Data represent mean±s.d. (n=4).

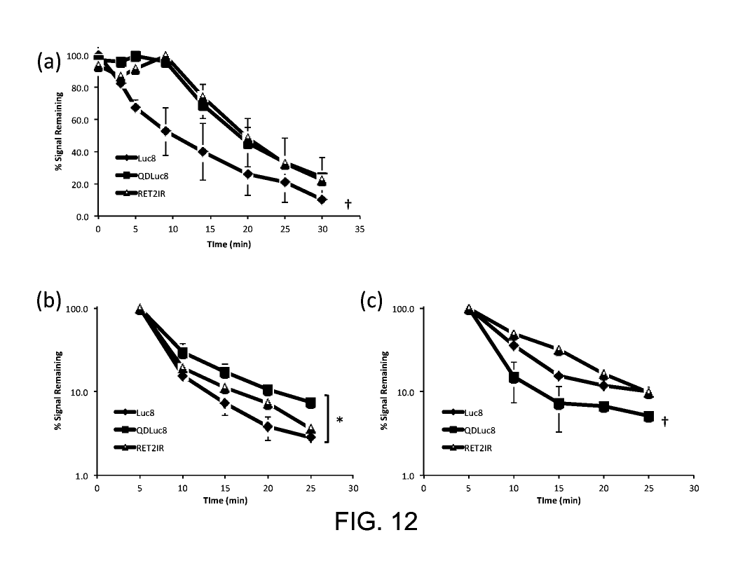

FIG. 12 illustrates the change luminescence was followed over time for Luc8 (blue diamonds), QDLuc8 (red squares) and RET₂IR (green triangles) (a) in vitro, and in vivo with (b) subcutaneous and (c) whole animal probe depth penetration. All data points represent mean±s.d. (n=4). * Significantly different luminescent signal decay over time (p<0.05). † Significantly faster luminescent signal decay over time relative to all other groups (p<0.05).

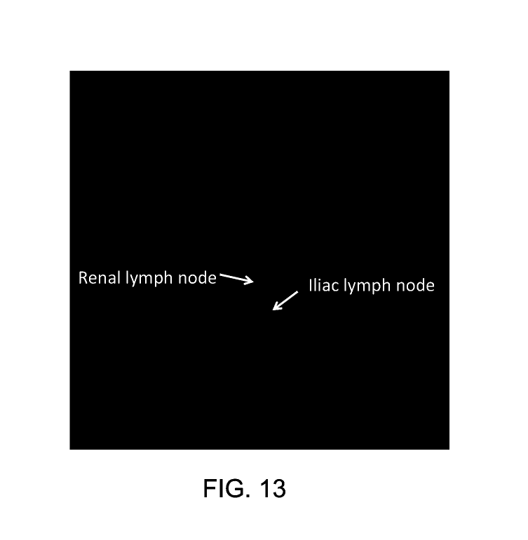

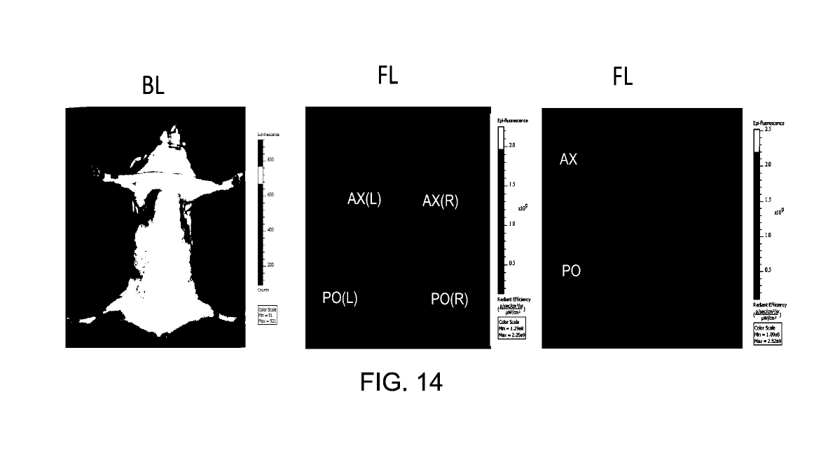

FIG. 13 illustrates the fluorescence imaging of the same mouse in FIG. 3a after the removal of internal organs. Both renal and iliac lymph nodes are clearly visualized. This is a representative of a total of 6 mice imaged.

FIG. 14 illustrates bioluminescence and fluorescence imaging of the same mouse in FIG. 3e 10 days after intradermal injection of RET₂IR NPs (2 μg) at four paws. (a) Bioluminescence imaging. (b) Fluorescence imaging; Superficial skin was removed before imaging but peritoneum intact. (c) Fluorescence imaging with the surgically opened mouse and excised axillary (AX) and popliteal (PO) lymph nodes. This is a representative of a total of 3 mice imaged.

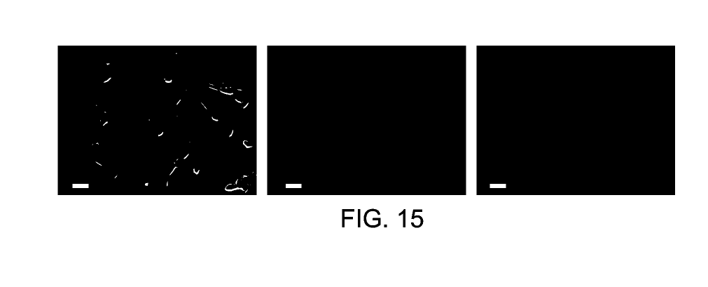

FIG. 15 illustrates live imaging of U87MG cells in vitro incubated with RET₁IR@cRGD NPs (4 μg) for 8 h. Left: bright field; middle: NIR fluorescence; right: overlay of NIR fluorescence and DAPI. The lack of overlapping between the NIR and DAPI blue fluorescence showed that the NPs were inside cells but not localized in the cell nucleus after 8 h incubation. Scale bar: 20 μm.

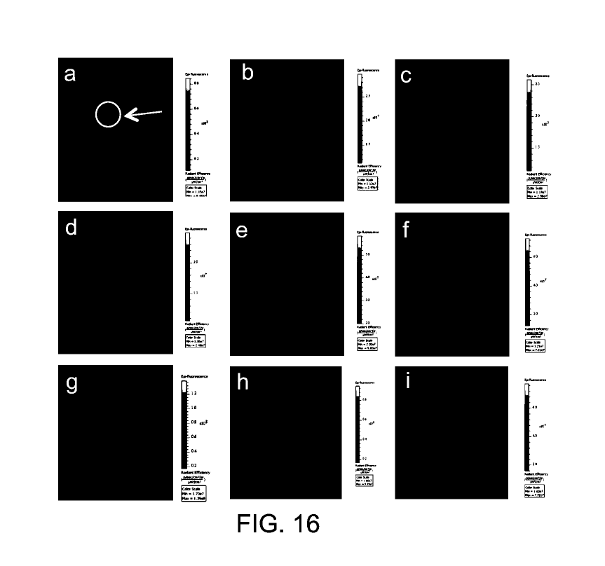

FIG. 16 illustrates time-dependent in vivo fluorescence imaging of a U87MG tumor-bearing mouse (indicated by white arrow; tumor size at 0.8 cm in average diameter) injected with RET₁IR@cRGD NPs (˜50 μg). Autofluorescence is coded in green and the unmixed NP signal in red. (a) 5 min, (b) 20 min, (c) 40 min, (d) 1 h, (e) 2 h, (f) 4 h, (g) 6 h, (h) 24 h, (i) 48 h. This is a representative of a total of 3 mice imaged.

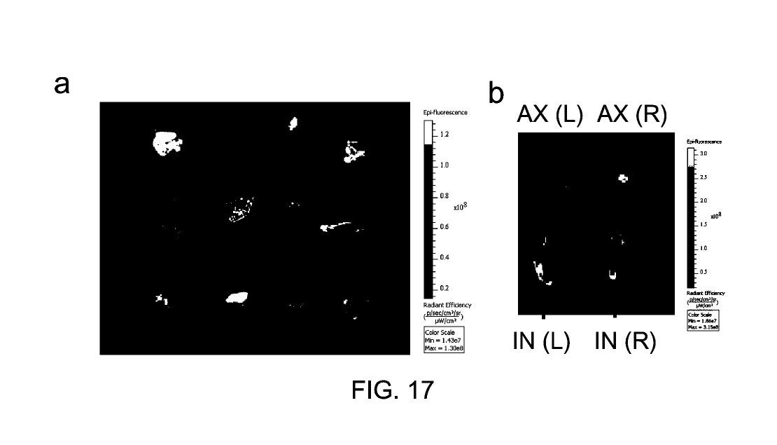

FIG. 17 illustrates fluorescence images of excised mouse organs and lymph nodes after 48 h injection of RET₁IR@cRGD NPs. (a) Organs: 1 brain, 2 tumor, 3 spleen, 4 pancreas, 5 kidney, 6 lung, 7 heart, 8 bone, 9 liver, 10 muscle, 11 skin, 12 stomach. (b) Lymph nodes: AX, axillary lymph node; IN, inguinal lymph node; L, left side; R, right side of the mouse body. This is a representative of a total of 3 mice imaged.

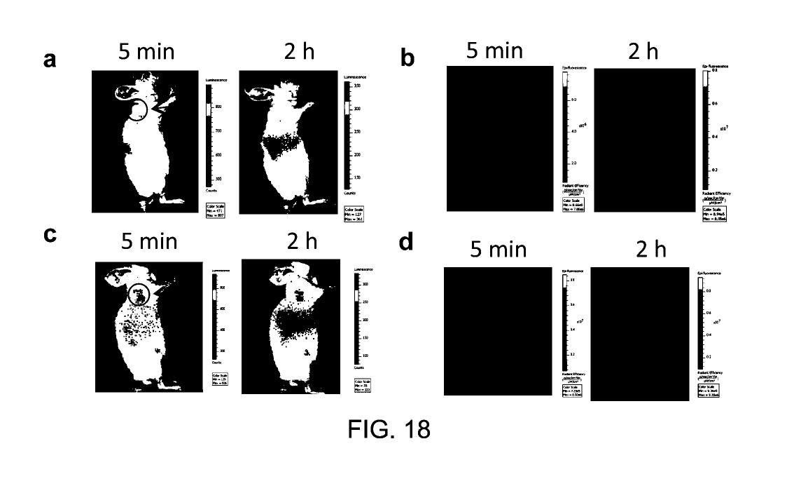

FIG. 18 illustrates in vivo bioluminescence (a, c) and fluorescence (b, d) imaging of U87MG tumors in mice. Tumors are marked by a red arrow and circle. (a, b) Mouse with a tumor of ˜5 mm in diameter was tail-vein co-injected with RET₂IR@cRGD (˜20 μg) and free cRGD (300 μg). This is a representative of a total of n=3 mice imaged. (c, d) Mouse was tail-vein injected with RET₂IR@cRGD (˜20 μg); tumor size is ˜3 mm in diameter. The bioluminescence acquisition time was 15 s for the 5 min images and 60 s for the 2 h images. This is a representative of a total of 3 mice imaged.

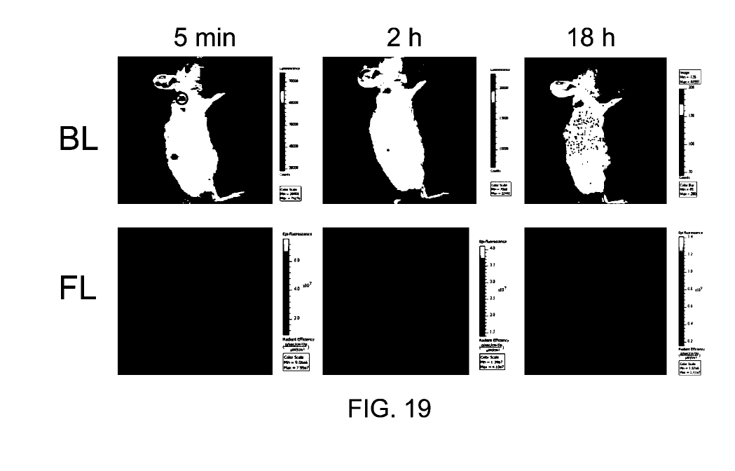

FIG. 19 illustrates in vivo bioluminescence and fluorescence imaging of small U87MG tumors in mice with RET₂IR@cRGD. Tumors are marked by a red arrow and circle, and tumor size is 2-3 mm in diameter. Each mouse was tail-vein injected RET₂IR@cRGD NPs (˜50 μg). The bioluminescence acquisition time from left to right: 15 s (5 min), 15 s (2 h), and 1 min (18 h). This is a representative of a total of 3 mice imaged.

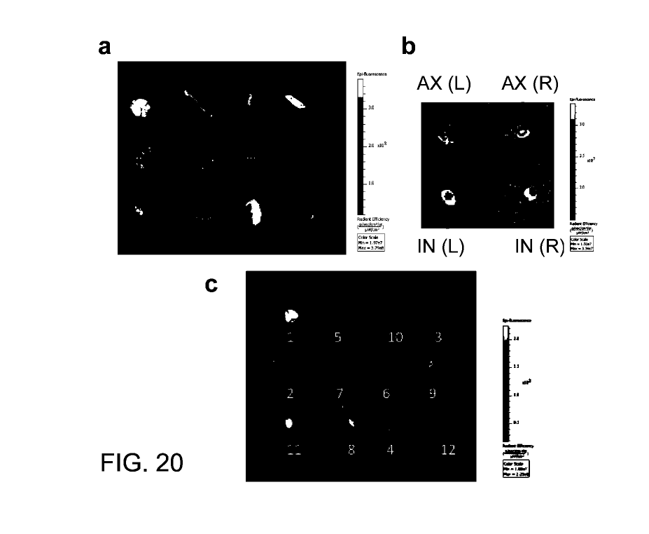

FIG. 20 illustrates fluorescence imaging of excised mouse organs and lymph nodes 48 h (a, b) or one week (c) after injection of RET₂IR@cRGD NPs. Organs in (a) and (c): 1 brain, 2 bone, 3 muscle, 4 skin, 5 kidneys, 6 lung, 7 heart, 8 spleen, 9 pancreas, 10 stomach, 11 liver, 12 tumor. (b) Lymph nodes: AX, axillary lymph nodes; IN, inguinal lymph nodes; L, left; R, right side of the mouse body. This is a representative of a total of 3 mice imaged.

References, each of which is incorporated herein by reference:
1. Ntziachristos, V., Ripoll, J., Wang, L. H. V. & Weissieder, R. Looking and listening to light: the evolution of whole-body photonic imaging. Nat. Biotechnol. 23, 313-320 (2005).
2. McDonald, D. M. & Chovke, P. L. Imaging of angiogenesis: from microscope to clinic. Nat. Med. 9, 713-725 (2003).
3. Contag, C. H. & Bachmann, M. H. Advances in vivo bioluminescence imaging of gene expression. Annu. Rev. Biomed. Eng. 4, 235-260 (2002).
4. Wagnieres, G. A., Star, W. M. & Wilson, B. C. In vivo fluorescence spectroscopy and imaging for oncological applications. Photochem. & Photobiol. 68, 603-632 (1998).
5. van Dam, G. M. et al. Intraoperative tumor-specific fluorescence imaging in ovarian cancer by folate receptor-alpha targeting: first in-human results. Nat. Med. 17, 1315-1319 (2011).
6. Frangioni, J. V. In vivo near-infrared fluorescence imaging. Curr. Opin. Chem. Biol. 5, 626-634 (2003).
7. So, M.-K., Xu, C., Loening, A. M., Gambhir, S. S. & Rao, J. Self-illuminating quantum dot conjugates for in vivo imaging. Nat. Biotechnol. 24, 339-343 (2006).
8. So, M.-K., Loening, A. M., Gambhir, S. S. & Rao, J. Creating self-illuminating quantum dot conjugates. Nat. Protocols 1, 1160-1164 (2006).
9. Wu, C., Kawasaki, K., Ohgiya, S. & Ohmiya, Y. Chemical studies on the BRET system between the bioluminescence of Cypridina and quantum dots. Photochem. Photobiol. Sci. 10, 1531-1534 (2011).
10. Du, J. et al. Quantum-dot-decorated robust transductable bioluminescent nanocapsules. J. Am. Chem. Soc. 132, 12780-12781 (2010).
11. Bacart, J., Corbel, C., Jockers, R., Bach, S. & Couturier, C. The BRET technology and its application to screening assays. Biotechnol. J. 3, 311-324 (2008).
12. Yao, H., Zhang, Y., Xiao, F., Xia, Z. & Rao, J. Quantum dot/bioluminescence resonance energy transfer based highly sensitive detection of proteases. Angew. Chem. Int. Ed. 46, 4346-4349 (2007).
13. Xia, Z. et al. Multiplex detection of protease activity with quantum dot nanosensors prepared by intein-mediated specific bioconjugation. Anal. Chem. 80, 8649-8655 (2008).
14. Cissell, K. A., Campbell, S. & Deo, S. K. Rapid, single-step nucleic acid detection. Anal. Bioanal. Chem. 391, 2577-2581 (2008).
15. Kosakaa, N., et al. Self-illuminating in vivo lymphatic imaging using a bioluminescence resonance energy transfer quantum dot nano-particle. Contrast Media Mol. Imaging 6, 55-59 (2011).
16. Xing, Y., So, M.-K., Koh, A. L., Sinclair, R. & Rao, J. Improved QD-BRET conjugates for detection and imaging. Biochem. Biophy. Res. Comm. 372, 388-394 (2008).
17. Schipper, M. L. et al. Particle size, surface coating, and PEGylation influence the biodistribution of quantum dots in living mice. Small 5, 126-134 (2009).
18. Schipper, M. L. et al. MicroPET-based biodistribution of quantum dots in living mice. J. Nucl. Med. 48, 1511-1518 (2007).
19. Pecher, J. & Mecking, S, Nanoparticles of conjugated polymers. Chem. Rev. 110, 6260-6279 (2010).
20. Rahim, N. A. A. et al. Conjugated polymer nanoparticles for two-photon imaging of endothelial cells in a tissue model. Adv. Mater. 21, 3492-3496 (2009).
21. Wu, C., Bull, B., Szymanski, C., K. Christensen, K. & McNeill, J. Multicolor conjugated polymer dots for biological fluorescence imaging. ACS Nano 2, 2415-2423 (2008).
22. Pu, K. Y., Li, K., Shi, J. B. & Liu, B. Fluorescent single-molecular core-shell nanospheres of hyperbranched conjugated polyelectrolyte for live-cell imaging. Chem. Mater. 21, 3816-3822 (2009).
23. Tang, H, Xing, C., Liu, L., Yang, Q. & Wang, S. Synthesis of amphiphilic polythiophene for cell imaging and monitoring the cellular distribution of a cisplatin anticancer drug. Small 7, 1464-1470 (2011).
24. Wu, C. et al. Bioconjugation of ultrabright semiconducting polymer dots for specific cellular targeting. J. Am. Chem. Soc. 132, 15410-15417 (2010).
25. Loening, A. M., Fenn, T. D., Wu, A. M. & Gambhir, S. S. Consensus guided mutagenesis of Renilla luciferase yields enhanced stability and light output. Protein Eng. Des. Sel. 19, 391-400 (2006).
26. Ruoslahti E. RGD and other recognition sequences for integrins. Annu. Rev. Cell Dev. Biol. 12, 697-715 (1996).
27. Hood, J. D. & Cheresh, D. A. Role of integrins in cell invasion and migration. Nat. Rev. Cancer 2, 91-100 (2002).
28. Xiong, J. P. et al. Crystal structure of the extracellular segment of integrin αᵥβ₃ in complex with an Arg-Gly-Asp ligand. Science 296, 151-155 (2002).
29. Ye, Y., Bloch, S., Xu, B. & Achilefu, S. Design, synthesis, and evaluation of near infrared fluorescent multimeric RGD peptides for targeting tumors. J. Med. Chem. 49, 2268-2275 (2006).
30. Jin, Y. H., Ye, F. M., Zeigler, M., Wu, C. F. & Chiu, D. T. Near-infrared fluorescent dye-doped semiconducting polymer dots. ACS Nano 5, 1468-1475 (2011).
31. Kim, S. et al. Near-infrared fluorescent type II quantum dots for sentinel lymph node mapping. Nat. Biotechnol. 22, 93-97 (2004).
32. Kobayashi, H. et al. Simultaneous multicolor imaging of five different lymphatic basins using quantum dots. Nano Lett. 7, 1711-1716 (2007).
33. Kim, S. et al. Conjugated polymer nanoparticles for biomedical in vivo imaging. Chem. Comm. 46, 1617-1619 (2010).
34. Harisinghani, M. G. et al. Noninvasive detection of clinically occult lymph-node metastases in prostate cancer. New Eng. Med. 348, 2491-2499 (2003).
35. Tanaka, E. et al. Image-guided oncologic surgery using invisible light: completed pre-clinical development for sentinel lymph node mapping. Ann. Surg. Oncol. 13, 1671-1681 (2006).
36. Ballou, B. et al. Noninvasive imaging of quantum dots in mice. Bioconjugate Chem. 15, 79-86 (2004).
37. Howes, P. et al. Phospholipid encapsulated semiconducting polymer nanoparticles: their use in cell imaging and protein attachment. J. Am. Chem. Soc. 132, 3989-3996 (2010).
38. Shuhendler, A. J. et al. Hybrid quantum dot-fatty ester stealth nanoparticles: toward clinically relevant in vivo optical imaging of deep tissue. ACS Nano 5, 1958-1966 (2011).

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to units and measuring techniques corresponding to the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

(Rluc)
SEQ ID NO: 1
MTSKVYDPEQ RKRMITGPQW WARCKQMNVL DSFINYYDSE
KHAENAVIFL HGNAASSYLW RHVVPHIEPV ARCIIPDLIG
MGKSGKSGNG SYRLLDHYKY LTAWFELLNL PKKIIFVGHD
WGACLAFHYS YEHQDKIKAI VHAESVVDVI ESWDEWPDIE
EDIALIKSEE GEKMVLENNF FVETMLPSKI MRKLEPEEFA
AYLEPFKEKG EVRRPTLSWP REIPLVKGGK PDVVQIVRNY
NAYLRASDDL PKMFIESDPG FFSNAIVEGA KKFPNTEFVK
VKGLHFSQED APDEMGKYIK SFVERVLKNE Q
(Rluc8)
SEQ ID NO: 2
MASKVYDPEQ RKRMITGPQW WARCKQMNVL DSFINYYDSE
KHAENAVIFL HGNATSSYLW RHVVPHIEPV ARCIIPDLIG
MGKSGKSGNG SYRLLDHYKY LTAWFELLNL PKKIIFVGHD
WGAALAFHYA YEHQDRIKAI VHMESVVDVI ESWDEWPDIE
EDIALIKSEE GEKMVLENNF FVETVLPSKI MRKLEPEEFA
AYLEPFKEKG EVRRPTLSWP REIPLVKGGK PDVVQIVRNY
NAYLRASDDL PKLFIESDPG FFSNAIVEGA KKFPNTEFVK
VKGLHFLQED APDEMGKYIK SFVERVLKNE Q

We claim the following:

1. A conjugate system, comprising: a BRET-FRET NIR conjugate and a bioluminescence initiating compound, wherein the BRET-FRET NIR conjugate includes a particle that includes a bioluminescence donor molecule and a NIR dye, wherein the particle includes a semiconductor polymer as part of the particle matrix, wherein the bioluminescence donor molecule is attached to the surface of the particle, wherein each of the NIR dyes are disposed within, attached to, or a combination thereof, the surface of the particle, wherein the bioluminescence donor molecule and the bioluminescence initiating compound interact to produce a bioluminescent energy, wherein the bioluminescent energy is absorbed by the semiconductor polymer and the semiconductor polymer produces fluorescent energy in response to the non-radiative transfer of the bioluminescent energy from the bioluminescence donor molecule to the semiconductor polymer, wherein the NIR dye absorbs the fluorescent energy and emits NIR energy in response to the energy transfer of the fluorescent energy from the semiconductor polymer to the NIR dye.

2. The system of claim 1, wherein the bioluminescence donor molecule is a Luciferase protein.

3. The system of claim 1, wherein the bioluminescence donor molecule is selected from a Renilla Luciferase protein, a mutated Renilla Luciferase protein, and a combination thereof.

4. The system of claim 1, wherein the semiconductor polymer is selected from the group consisting of: poly[2-methoxy-5-(2′-ethylhexyloxy)-p-phenylene vinylene], poly[2-methoxy-5-(2-ethylhexyloxy)-1,4-(1-cyanovinylene-1,4-phenylene)], poly[(9,9-dioctyl-2,7-divinylenefluorenylene)-alt-co-(9,10-anthracene)], poly[{9,9-dihexyl-2,7-bis(1-cyanovinylene)fluorenylene}-alt-co-{2,5-bis(N,N′-diphenylamino)-1,4-phenylene}], poly[(9,9-dioctylfluorenyl-2,7-diyl)-alt-(benzo[2,1,3]thiadiazol-4,7-diyl)], poly[2,7-(9,9-di-octyl-fluorene)-alt-4,7-bis(thiophen-2-yl)benzo-2,1,3-thiadiazole], poly[2,6-(4,4′-bis(2-ethylhexyl)dithieno[3,2-b:2′,3′-d]silole)-alt-4,7(2,1,3-benzothiadiazole)], poly[2,7-(9,9-di-octyl-fluorene)-alt-4,7-bis(thiophen-2-yl)benzo-2,1,3-thiadiazole], poly[N-9′-heptadecanyl-2,7-carbazole-alt-5,5-(4′,7′-di-2-thienyl-2′,1′,3′-benzothiadiazole)], poly[3-hexylthiophene-2,5-diyl], and poly[2,5-bis(3-dodecylthiophen-2-yl)thieno[3,2-b]thiophene], combinations of these.

5. The system of claim 1, wherein the NIR dye is selected from 4,4-difluoro-4-bora-3a,4a-diaza-s-indacene and derivatives thereof.

6. The system of claim 1, wherein the particle includes an agent.

7. The system of claim 6, wherein the agent has an affinity for a target, where the target is selected from: a compound, a polypeptide, a polynucleotide, an antibody, an antigen, a hapten, a cell type, a tissue type functional group, a tissue type, and combinations thereof.

8. The system of claim 7, wherein the particle includes another agent, wherein the agent is effective at treating a disease.

9. The system of claim 1, wherein the particle includes an agent, wherein the agent is effective at treating a disease.

10. The system of claim 1, wherein the bioluminescence initiating compound is selected from coelenterazine, analogs, and functional derivatives thereof, and D-luciferin analogs, and functional derivatives thereof.

11. The system of claim 1, wherein the particle is coated with or includes moieties that are bound to the bioluminescence donor molecule.

12. The system of claim 1, wherein the particle is coated with an agent that improves biocompatibility relative to the particle not including the agent.

13. A conjugate, comprising: a BRET-FRET NIR conjugate, wherein the BRET-FRET NIR conjugate includes a particle that includes a bioluminescence donor molecule and a NIR dye, wherein the particle includes a semiconductor polymer as part of the particle matrix, wherein the bioluminescence donor molecule is attached to the surface of the particle, wherein each of the NIR dyes are disposed within, attached to, or a combination thereof, the surface of the particle.

14. The conjugate of claim 13, wherein the bioluminescence donor molecule is a Luciferase protein.

15. The conjugate of claim 13, wherein the bioluminescence donor molecule is selected from a Renilla Luciferase protein, a mutated Renilla Luciferase protein, and a combination thereof.

16. The conjugate of claim 13, wherein the semiconductor polymer is selected from the group consisting of: poly[2-methoxy-5-(2′-ethylhexyloxy)-p-phenylene vinylene], poly[2-methoxy-5-(2-ethylhexyloxy)-1,4-(1-cyanovinylene-1,4-phenylene)], poly[(9,9-dioctyl-2,7-divinylenefluorenylene)-alt-co-(9,10-anthracene)], poly[{9,9-dihexyl-2,7-bis(1-cyanovinylene)fluorenylene}-alt-co-{2,5-bis(N,N′-diphenylamino)-1,4-phenylene}], poly[(9,9-dioctylfluorenyl-2,7-diyl)-alt-(benzo[2,1,3]thiadiazol-4,7-diyl)], poly[2,7-(9,9-di-octyl-fluorene)-alt-4,7-bis(thiophen-2-yl)benzo-2,1,3-thiadiazole], poly[2,6-(4,4′-bis(2-ethylhexyl)dithieno[3,2-b:2′,3′-d]silole)-alt-4,7(2,1,3-benzothiadiazole)], poly[2,7-(9,9-di-octyl-fluorene)-alt-4,7-bis(thiophen-2-yl)benzo-2,1,3-thiadiazole], poly[N-9′-heptadecanyl-2,7-carbazole-alt-5,5-(4′,7′-di-2-thienyl-2′,1′,3′-benzothiadiazole)], poly[3-hexylthiophene-2,5-diyl], and poly[2,5-bis(3-dodecylthiophen-2-yl)thieno[3,2-b]thiophene], combinations of these.

17. The conjugate of claim 13, wherein the NIR dye is selected from 4,4-difluoro-4-bora-3a,4a-diaza-s-indacene and derivatives thereof.

18. The conjugate of claim 13, wherein the particle includes an agent.

19. The conjugate of claim 18, wherein the agent has an affinity for a target, where the target is selected from: a compound, a polypeptide, a polynucleotide, an antibody, an antigen, a hapten, a cell type, a tissue type functional group, a tissue type, and combinations thereof.

20. The conjugate of claim 19, wherein the particle includes another agent, wherein the agent is effective at treating a disease.

21. The conjugate of claim 13, wherein the particle includes an agent, wherein the agent is effective at treating a disease.

22. A method of detecting a target, comprising:
providing a BRET-FRET NIR conjugate, wherein the BRET-FRET NIR conjugate includes a particle that includes a bioluminescence donor molecule and a NIR dye, wherein the particle includes a semiconductor polymer as part of the particle matrix, wherein the bioluminescence donor molecule is attached to the surface of the particle, wherein each of the NIR dyes are disposed within, attached to, or a combination thereof, the surface of the particle;
introducing the BRET-FRET NIR conjugate to a host;
introducing a bioluminescence initiating compound to the host; and
determining the presence and location of the target corresponding to the agent by detecting the BRET-FRET NIR conjugate upon interaction with the bioluminescence initiating compound.

23. The method of claim 22, wherein determining includes detecting a radiation emission from the NIR dye without the use of an external illumination source.

24. The method of claim 22, wherein determining includes determining the presence and location of the target in the host in vivo.

25. The method of claim 22, wherein determining includes determining the presence and location of the target in the host in vitro.

26. A method of detecting a target, comprising:
providing a BRET-FRET NIR conjugate, wherein the BRET-FRET NIR conjugate includes a particle that includes a bioluminescence donor molecule and a NIR dye, wherein the particle includes a semiconductor polymer as part of the particle matrix, wherein the bioluminescence donor molecule is attached to the surface of the particle, wherein each of the NIR dyes are disposed within, attached to, or a combination thereof, the surface of the particle;
introducing the BRET-FRET NIR conjugate to a system;
introducing a bioluminescence initiating compound to the system; and
determining the presence of the target corresponding to the agent by detecting the BRET-FRET NIR conjugate upon interaction with the bioluminescence initiating compound.

27. The method of claim 26, where the system is a biological assay.
